(19) United States
(12) Patent Application Publication
(10) Pub. No.: US 2015/0025052 A1
(43) Pub. Date: Jan. 22, 2015

(54) Compositions and Methods for Inhibiting HSP90/HSP70 Machinery

(71) Applicant: Georgia Regents Research Institute, Inc., Augusta, GA (US)

(72) Inventors: Ahmed Chadli, Evans, GA (US); Chaitanya Anil Patwardhan, Augusta, GA (US)

(21) Appl. No.: 14/333,172

(22) Filed: Jul. 16, 2014

Related U.S. Application Data
(60) Provisional application No. 61/846,856, filed on Jul. 16, 2013.

(51) Int. Cl. A61K 31/165; A61K 31/395

(52) U.S. Cl. CPC A61K 31/165; A61K 31/395
USPC 514/183; 435/375

(57) ABSTRACT

Pharmaceutical compositions including an effective amount of a capsaicin and inhibitors of Hsp90 to decrease or inhibit Hsp70 and Hsp90 chaperone pathways in cells are disclosed. Methods of inhibiting the Hsp70 and Hsp90 chaperone pathways including contacting cells expressing the Hsp70/Hsp90 complex with an effective amount of a capsaicin in combination with inhibitors of Hsp90 to decrease or inhibit the Hsp70 and Hsp90 chaperone pathways are provided. The methods can reduce the viability of target cells, for example, by increasing apoptosis or pro-apoptotic pathways. In preferred embodiments, the methods reduce or do not increase Hsp70, Hsp90, Hsp40, or HOP expression; reduce or do not increase heat shock response; reduce or do not increase pro-survival pathways in cells. Methods of treating cancer and other diseases using the disclosed compositions and methods are provided.

### CROSS REFERENCE TO RELATED APPLICATIONS

This application claims priority to and benefit of U.S. Provisional Patent Application No. 61/846,856 filed on Jul. 16, 2013, which is incorporated by reference in its entirety.

### STATEMENT REGARDING FEDERALLY SPONSORED RESEARCH

This invention was made with government support under Grant GM102443-01 awarded by the National Institutes of Health. The government has certain rights in the invention

### FIELD OF THE INVENTION

The present invention relates to the treatment of cancer and in particular to the application of capsaicin and its derivatives in combination with inhibitors of the heat shock protein Hsp90 pathway for the enhanced killing of tumor cells.

### BACKGROUND OF THE INVENTION

Hsp90 is currently considered a promising therapeutic target for cancer and over 40 clinical trials in phases I-III with inhibitors of Hsp90 are ongoing worldwide (Neckers, et al., Clinical cancer research, 18:64-76 (2012)). Most of these inhibitors target the N-terminal ATP binding site to inactivate the ATPase activity of Hsp90, causing proteasomal degradation of its client proteins. Unfortunately many N-terminus inhibitors, such as geldanamycin or its analog 17-AAG induce a heat-shock response and overexpression of apoptosis inhibitor proteins Hsp70 and Hsp27, which are thought to contribute to the modest outcomes of these inhibitors observed in the clinic (Whitesell, et al., Nature Reviews Cancer, 5:761-772 (2005); Workman, Cancer Letters 206:149-157 (2004); Neckers, et al., Clinical Cancer Research, 18:64-76 (2012); Davenport, et al., Leukemia UK, 24:1804-1807 (2010)).

The inhibition of Hsp70 can sensitize tumor cells to chemotherapy with currently available Hsp90 inhibitors. Simultaneous inactivation of both Hsp70 and Hsp90 would deliver a combinatorial attack on multiple signaling pathways, enhancing the efficacy of the available Hsp90 inhibitors and leading to more efficient killing of cancer cells (Pratt and Toft, Exp. Bio. Med., 228:111-133 (2003)).

Therefore, it is an object of the invention to provide compositions and methods for the treatment of cancer through inhibitors of Hsp70 in combination with inhibitors of Hsp90.

It is another object of the invention to provide compositions and methods for the selective inhibition of Hsp70.

It is yet another object of the invention to provide a method for identifying or screening for selective inhibitors of Hsp70.

It is still another embodiment or the invention to provide compositions and methods for inhibiting or reducing cell proliferation, in particular tumor cell proliferation.

### SUMMARY OF THE INVENTION

Compositions and methods for inhibiting the Hsp90 machinery in cells, preferably cancer or tumor cells, are provided. It has been discovered that capsaicin or analogs thereof, alter the Hsp70 multi-chaperone complexes in cells and destabilize Hsp90/Hsp70 client proteins. Because Hsp90 molecular chaperone machinery is a key modulator of the cancer phenotype, the disclosed compositions and methods are useful for treating cancer, a symptom thereof, or for inhibiting or reducing cellular proliferation due to cancer.

Capsaicin can be used alone or in combination with a second agent to inhibit the Hsp90 machinery in cells, particularly in dividing cells such as cancer or tumor cells. Existing Hsp90 inhibitors used in the treatment of cancer have the unpleasant side-effect of upregulating Hsp70. Hsp70 is an anti-apoptotic protein, and upregulating Hsp70 reduces the overall effect of Hsp90 inhibitors. Therefore, one embodiment provides a composition including an effective amount of an Hsp90 inhibitor to inhibit Hsp90 in combination with an effective amount of Hsp70 inhibitor to inhibit Hsp70. In a preferred embodiment, the inhibitor of Hsp70 selectively inhibits Hsp70 (inducible isoform) and not Hsc70 (constitutive isoform). In a more preferred embodiment, the Hsp70 inhibitor is capsaicin or an analog thereof.

Degradation of Hsp90/Hsp70 complexes by capsaicin provides a means to overcome the potential complications associated with increasing pro-survival signaling that results from Hsp70. Thus, the use of capsaicin to abrogate protective anti-apoptotic mechanisms provided by Hsp70 is disclosed. Combinations of capsaicin and Hsp90 inhibitors to exert a cytotoxic effect, leading to a more efficient combinatorial antitumor therapy are also described.

Pharmaceutical compositions including an effective amount of a capsaicin, a synthetic capsaicin, or a derivative, analog or prodrug, or a pharmacologically active salt thereof and one or more inhibitors of Hsp90 to reduce, decrease, or inhibit the Hsp70 and Hsp90 chaperone pathways in cells compared to a control are provided.

In preferred embodiments one inhibitor of the Hsp90 pathway is tanespimycin (17-AAG). In some embodiments the pharmaceutical composition includes a delivery vehicle, such as a microparticle or a nanoparticle. In some embodiments the composition includes a pharmaceutically acceptable carrier.

Methods of inhibiting the Hsp70 and Hsp90 chaperone pathways including contacting one or more cells expressing Hsp70 and/or Hsp90 with an effective amount of a capsaicin, a synthetic capsaicin, or a derivative, analog or prodrug, or a pharmacologically active salt thereof in combination with one or more inhibitors of Hsp90 to decrease or inhibit the Hsp70 and Hsp90 chaperone pathways in the cells compared to control cells are also provided. Typically the cells are under stress or transforming pressure, are diseased or pathogenic. In some embodiments the capsaicin, synthetic capsaicin, or derivative, analog or prodrug, or pharmacologically active salt thereof in combination with one or more inhibitors of Hsp90 reduces the viability of the cells or increases apoptosis of the cells.

In some embodiments the naturally occurring capsaicin, synthetic capsaicin, or derivative, analog or prodrug, or pharmacologically active salt thereof in combination with one or more inhibitors of Hsp90 prevent the formation of, or increase the degradation of Hsp70/Hsp90 complexes optionally including one or more co-chaperones or client proteins, such as AKT, pAKT and CDK4, ILK, Her2, Her3 or HOP. In some embodiments the capsaicin, synthetic capsaicin, or derivative, analog or prodrug, or pharmacologically active salt thereof in combination with one or more inhibitors of Hsp90 reduces or inhibits Hsp70 or Hsp90-mediated folding, activation, assembly, or function of client proteins.

Methods of treating disease in a subject typically include administering to a subject with a disease an effective amount of capsaicin, synthetic capsaicin, or a derivative, analog or prodrug, or pharmacologically active salt thereof in combination with one or more inhibitors of Hsp90 to reduce or inhibit one or more symptoms of the disease are also provided. In some embodiments the disease to be treated is cancer. In preferred embodiments the cancer to be treated is triple-negative breast cancer or hormone-refractory prostate cancer.

In some embodiments capsaicin, or a derivative, analog or prodrug, or pharmacologically active salt thereof in combination with one or more inhibitors of Hsp90 is administered to the subject in combination with one or more additional therapeutic agents.

Methods for inhibiting the Hsp70 chaperone pathway including administering an effective amount of a naturally occurring capsaicin, a synthetic capsaicin, or a derivative, analog or prodrug, or a pharmacologically active salt thereof to reduce, decrease, or inhibit the Hsp70 chaperone pathway in cells compared to a control are also disclosed. The cells are typically characterized by expression of the Hsp70 or Hsp90 complex, or are under stress or transforming pressure. In some embodiments, the methods prevent the formation of Hsp70 complexes optionally including one or more co-chaperones. In some embodiments, the methods increase the degradation of Hsp70 complexes optionally including one or more co-chaperones or client proteins such as Hsp90 and HOP; reduce or inhibit Hsp70-mediated folding, activation, assembly, or function of proteins or denatured proteins or a combination thereof. The methods can reduce the viability of the target cells, for example, by increasing apoptosis or pro-apoptotic pathways. In preferred embodiments, the methods reduce or do not increase Hsp70, Hsp90, Hsp24, Hsp40, or HOP expression; reduce or do not increase the heat shock response; reduce or do not increase pro-survival pathways in the cells. In preferred embodiments, the heat shock response in cells treated with an Hsp90 inhibitor and a capsaicin, synthetic capsaicin, or derivative, analog or prodrug, or pharmacologically active salt thereof, is less than the heat shock response in cells treated with an Hsp90 inhibitor in the absence of the capsaicin.

The methods can include administering to the subject a second therapeutic agent, for example a chemotherapeutic agent. Methods of treating diseases and conditions such as cancer, inflammatory diseases or disorders, neurodegenerative diseases, and infectious diseases using the disclosed compositions and methods are also disclosed.

Methods for identifying or screening for Hsp90/Hsp70 inhibitors are also provided.

### BRIEF DESCRIPTION OF THE DRAWINGS

FIG. 1 is a histogram showing the binding of [3H]-Progesterone (measured in cpm) to the progesterone receptor following refolding by Hsp proteins in the presence of a panel of inhibitor compounds, including capsaicin (indicated by an arrow at the base).

FIGS. 2A-2F are autoradiographs of composite images of Western immuno-staining blots. FIG. 2A shows the Hsp90 client proteins HER2, GR, PRB, CDK4, Chk1, Raf-A, Akt and ILK, and the chaperones Hsp70i, Hsc70, HDJ2 (Hsp40), Hsp27, Hsp90α and Hsp90β in the presence of DMSO, 2.5 μM 17AAG or 50, 100 or 200 μM capsaicin (Comp C) in HeLa-PR_{B} cells. FIG. 2B shows the Hsp90 client proteins HER2, GR, CDK4, Chk1, Raf-A, Akt and ILK, and the chaperones Hsp70i, Hsc70, HDJ2 (Hsp40), Hsp27, Hsp90α and Hsp90β in the presence of DMSO, 2.5 μM 17AAG, or 50 or 100 μM capsaicin (Comp C) in MCF7 cells. FIG. 2C shows the Hsp90 client proteins AR, Akt, Raf-A and CDK4, and the chaperones Hsp70i, Hsc70, Hsp27, Hsp90β and p23 in the presence of DMSO, 2.5 μM 17AAG or 50, 100 or 200 μM and LNCaP cells. FIGS. 2D-E show the Hsp90 client proteins HER2, GR, PRB, CDK4, Chk1, Raf-A, Akt and ILK, and the chaperones Hsp70, 2;3, Hsc70, HOP, HDJ2 (Hsp40), p23, Hsp90α and Hsp90β in the presence of DMSO, 2.5 μM 17AAG or 50, 100 or 200 μM capsaicin in HeLa cells (FIG. 2D) or MCF7 cells (FIG. 2E), respectively. In all blots, 2.5 μM 17-AAG was used as a positive control for cellular degradation of several kinases and hormone receptor clients of Hsp90, Beta actin (β-actin) was used a loading control. FIG. 2F is an autoradiograph of a Western blot showing capsaicin altered Hsp70 multi-chaperone complexes in cells. LNCaP cells were treated with 200 μM capsaicin for 6 h and then lysed, and Hsp70 complexes were pulled down using an antibody against Hsp70 on Protein A-Sepharose beads. Western blotting was performed for Hsp70, Hsp40 (HDJ-2), and Hsp90β. Capsaicin reduced Hsp40 and Hsp90 binding to Hsp70. Mouse IgG was used as a control.

FIG. 3A is a line graph of absorbance at 495 nm (arbitrary units) versus Capsaicin dose (μM) of Hs578Bst and MCF7 cells treated with the indicated dose of capsaicin. FIG. 3B is a line graph of absorbance at 495 nm versus time of capsaicin treatment (hours) of Hs578Bst cells and MCF7 cells. Stippled line represents Hs578Bst cells and solid line represents MCF7 cells. FIGS. 3C-F are histograms showing colony number versus treatment with DMSO or Capsaicin in the following cell lines: LNCaP (FIG. 3C), MCF7 (FIG. 3D), HepG2 (FIG. 3E), and HeLa (FIG. 3F). Cells were treated with 200 μM capsaicin for 24 h. Adherent cells were then collected and plated (1000 cells per 10 cm dish) for further incubation. The standard deviation of three dishes is shown as error bars. The experiment was reproduced twice. FIG. 3G is an autoradiograph of cell lysates HeLa, MCF7 and LNCaP treated with 200 μM capsaicin for 24 h; cell lysates were made and Western blotted for p62/SQSTM1 and LC3II. β-actin was used as a loading control. LNCaP cells were treated with 50, 100 and 200 μM capsaicin for 24 h. this experiment was reproduced twice. FIG. 3H shows concentration-dependent induction of LC3B modification in LNCaP cells after 24 h treatment with 50, 100, 200 μM capsaicin. DMSO was used as a control.

FIG. 4A is a histogram showing the number of surviving HeLa PRB cells (×100,000) in the presence of DMSO, 2.5 μM 17AAG or 50, 100 or 200 μM capsaicin (Comp C). FIG. 4B is a histogram showing the number of surviving T47D cells in the presence of DMSO, 2.5 μM 17AAG or 50, 100 or 200 μM capsaicin (Comp C). FIG. 4C is a line graph of Absorbance at 495 nm of Hs578T normal mammary cells versus 0, 50, 100, 150 or 200 μM capsaicin (Compound C), 4 days (♦), 5 days (▪) and 7 days (▴) after treatment.

FIG. 5A is a histogram showing the ratio of Hsp70 mRNA relative to β-actin in the presence of DMSO, 100 μM or 200 μM capsaicin (Cap) after RT-PCR. FIG. 5B is a graph of fluorescence emission spectra for capsaicin, Hsp70, Hsp70 with capsaicin (Hsp70+C), Hsp90, Hsp90 with capsaicin (Hsp90+C), Hsp40 and Hsp40 with capsaicin (Hsp40+C), at wavelengths between 290 and 440 nm. All chaperones were tested at 15 μM and capsaicin at 7.5 μM. Proteins were incubated for 1 h at room temp in the presence or absence of capsaicin. An excitation wavelength of 266 nm was used.

FIG. 6A is an autoradiograph of composite images of Western immuno-staining blots, showing Hsp70 and GAPDH in MCF7 cells (top panels) and LNCaP cells (bottom panels), respectively, in the presence of DMSO or 100 nM 17AAG, or 50, 100 or 200 μM capsaicin (Compound C) for 24 h. GAPDH was used as loading control. FIG. 6B is a histogram showing the relative survival of LNCaP cells in the presence of DMSO, 800 nM 17AAG, 50 μM capsaicin (Comp C) and a mixture of 800 nM 17AAG with 50 μM capsaicin (Comp C).

FIGS. 7A-7D are data from RT-PCR of MCF7 cells and Western Blots of cells treated with either capsaicin (200 μM) or 17-AAG (200 nM) or in combination. DMSO was used as a negative control. The samples were supplemented with either MG132 (20 μM) or 3MA (5 mM) and further incubated for 6 h. FIG. 7A is an autoradiograph of cells using Hsp70 specific primers. Quantification of Hsp70 band intensity relative to β-actin loading control is shown in the lower panel. FIG. 7B is a histogram showing the relative mRNA of Hsp70/β-actin in the presence of DMSO, 17AAG, capsaicin (Comp C), 17AAG with capsaicin (17AAG+Comp C), DMSO with 5 mM 3MA (DMSO+3MA (5 mM)), 17AAG+3MA (5 mM), CompC+3MA (5 mM) and 17AAG+CompC+3MA. FIG. 7C is an autoradiograph of cells Western blotted for Hsp70 and LC3II. GAPDH was used as a loading control. Capsaicin blocks 17-AAG-mediated over-expression of Hsp70 and triggers lysosome-autophagy mediated degradation of Hsp70. Quantification of Hsp70 band intensity relative to GAPDH control is shown in the lower panel. FIG. 7D is a histogram of protein Hsp70/GAPDH (AU) for the cells treated in FIG. 7B.

FIGS. 8A-8F are histograms of percent Hormone binding activity (cpm) in the presence or absence of chemical compounds from NIH Clinical Collection drug library. PR complexes were reconstituted on a 96-well plate using a PR22 antibody and rabbit reticulocyte lysate (RRL) as the source of molecular chaperones and accessory proteins. Each bar represents percent hormone-binding activity of PR in presence or absence of chemical compounds. The first eight samples represent following internal controls: 1. Protein A alone, 2. Protein A+PR22, 3. Protein A+PR22+PR, 4. Protein A+PR22+PR+RRL, 5. Protein A+PR22+PR+RRL+17-AAG (20 μM), 6. Protein A+PR22+PR+RRL+Myrecetin (20 μM), 7. Protein A+PR22+PR+RRL+geldanamycin (20 μM), 8. Protein A+PR22+PR+RRL+gedunin (20 μM). PR22 is a monoclonal antibody against Avian PR. The remaining samples contain compounds from the NCP000685 plate (FIG. 8A), NCP001097 (FIG. 8B), NCP000200 (FIG. 8C), NCP000899 (FIG. 8D), NCP000998 (FIG. 8E), NCP001169 (FIG. 8F) used at 10 μM final concentration. Primary hits are represented with arrows on the x-axis Inhibitors and activators that are non-steroidal compounds are listed in Table 1. False positive hits (steroidal compounds) are listed in Table 2. The standard deviation of duplicate samples is shown as error bars.

FIG. 9A is a histogram of percent Hormone binding (cpm) in the presence or absence of chemical compounds. PR complexes were reconstituted on a 96-well plate as in FIG. 8. The first four wells are internal controls: 1. Protein A+PR22+PR, 2. Protein A+PR22+PR+RRL, 3. Protein A+PR22+PR+RRL+17-AAG (20 μM), 4. Protein A+PR22+PR+RRL+Myrecetin (20 μM). Compound hits from indicated plates were re-screened at 20 μM final concentration. The standard deviation of triplicate samples is shown as error bars. Plate no. 1. NCP000685, 2. NCP001097, 3. NCP000800, 5. NCP000998. Progesterone was used as a positive control. FIG. 9B provides the chemical structures of each of the hits shown in FIG. 9A. The corresponding names of these compounds are listed in Table. 1.

FIG. 10 provides the chemical structures of false positive hits obtained from the high-throughput screening of NIH Clinical Collection drug library. Chemical structures of these compounds resemble steroid receptor ligands. The chemical names of all of the compounds are listed in Table 2.

FIG. 11A is an autoradiograph of MCF7 and LNCaP cells treated with 50, 100 and 200 μM capsaicin, either alone or in combination with 100 nM 17-AAG for 24 h and Western blotted for Hsp70. Capsaicin prevented 17-AAG-induced upregulation of Hsp70 in both cell lines. DMSO was used as a negative control. GAPDH was used as a loading control. FIG. 11B is a histogram of MTT cell survival assays of MCF7 cells treated for 48 h with either 800 nM 17-AAG, various concentrations of capsaicin (12.5, 25 or 50 μM) or the combination. For MCF7 only 50 μM capsaicin was used alone or in combination with 800 nM 17-AAG. DMSO was used as a negative control. The standard deviation of quadruplet samples is shown as error bars. The experiment was repeated three times. FIG. 11C is the same as FIG. 11B except the cells are LNCaP cells.

FIG. 12 is an autoradiograph of LNCaP cells treated with 200 μM capsaicin for 6 h and then lysed and Western blotted for Hsp70, Hsp40 (HDJ-2), and Hsp90β. Capsaicin reduced Hsp40 and Hsp90 binding to Hsp70. Mouse IgG was used as a control.

### DETAILED DESCRIPTION OF THE INVENTION

### I. Definitions

The term “Hsp70” includes each member of the family of heat shock proteins having a mass of about 70 kilo Daltons. For example, in humans the highly conserved Hsp70 family includes the Hsc70 (heat shock cognate 70, Hsp73, HspA8) and Hsp70 (Hsp72, HspA1A/A1B) isoforms, as well as GRP78 (Hsp70-5), which is found in the endoplasmic reticulum, and Grp75 (Hsp70-9), which is found in the mitochondrial matrix. Hsp70 family proteins are also found in protozoa, such as PfHsp70 of P. falciparum, and in prokaryotes, such as DnaK of E. coli. An Hsp70 protein that is induced in a cell can be referred to as “Hsp70i”.

The terms “Hsp70 chaperone pathway” or “Hsp70 pathway” refer to cellular processes that involve the chaperoning activity of Hsp70.

The term “Hsp70 inhibitor” refers to an agent that reduces, decreases, or inhibits the expression or activity of Hsp70 or the Hsp70 chaperone pathway. The agent can inhibit Hsp70 directly or indirectly.

The term “Hsp90” includes each member of the family of heat shock proteins having a mass of about 90 kilo Daltons. For example, in humans the highly conserved Hsp90 family includes the cytosolic Hsp90alpha (Hsp90α) and Hsp90beta (Hsp90(3) isoforms, as well as GRP94, which is found in the endoplasmic reticulum, and Hsp75/TRAP1, which is found in the mitochondrial matrix.

The terms “Hsp90 chaperone pathway” or “Hsp90 pathway” refer to cellular processes that involve the chaperoning activity of Hsp90.

The term “Hsp90 inhibitor” refers to an agent that reduces, decreases, or inhibits the expression or activity of Hsp90 or the Hsp90 chaperone pathway. The agent can inhibit Hsp90 directly or indirectly.

The term “Capsaicin” refers to the capsaicinoid of Formula IV.

The term “effective amount” or “therapeutically effective amount” means a dosage sufficient to provide treatment of the disease state being treated or to otherwise provide a desired pharmacologic and/or physiologic effect. The precise dosage will vary according to a variety of factors such as subject-dependent variables (e.g., age, immune system health, etc.), the disease, and the treatment being effected.

The terms “individual,” “subject,” and “patient” are used interchangeably herein, and refer to a mammal, including, but not limited to, rodents, simians, humans, mammalian farm animals, mammalian sport animals, and mammalian pets.

The terms “treat”, “treatment” and “treating” refer to the reduction or amelioration of the progression, severity and/or duration of a disease or disorder, delay of the onset of a disease or disorder, or the amelioration of one or more symptoms (preferably, one or more discernible symptoms) of a disease or disorder, resulting from the administration of one or more therapies (e.g., one or more therapeutic agents such as a compound of the invention). The terms “treat”, “treatment” and “treating” also encompass the reduction of the risk of developing a disease or disorder, and the delay or inhibition of the recurrence of a disease or disorder.

The terms “reduce”, “inhibit” or “decrease” are used relative to a control. Controls are known in the art. For example a decrease response in a subject or cell treated with a compound is compared to a response in subject or cell that is not treated with the compound.

The term “pharmaceutically acceptable carrier” means a carrier combination of carrier ingredients approved by a regulatory agency of the Federal or a state government or listed in the U.S. Pharmacopoeia or other generally recognized pharmacopoeia for use in animals, mammals, and more particularly in humans. Non-limiting examples of pharmaceutically acceptable carriers include liquids, such as water and oils, including those of petroleum, animal, vegetable, or synthetic origin. Water is preferred vehicle when the compound of the invention is administered intravenously. Saline solutions and aqueous dextrose and glycerol solutions can also be employed as liquid vehicles, particularly for injectable solutions.

The term “in combination” refers to the use of more than one therapeutic agent. The use of the term “in combination” does not restrict the order in which said therapeutic agents are administered to a subject.

The term “17-AAG” refers to tanespimycin (17-N-allylamino-17-demethoxygeldanamycin), the derivative of the antibiotic geldanamycin that is an inhibitor of Hsp90.

“Localization Signal or Sequence or Domain or Ligand” or “Targeting Signal or Sequence or Domain or Ligand” are used interchangeably and refer to a signal that directs a molecule to a specific cell, tissue, organelle, or intracellular region. The signal can be polynucleotide, polypeptide, or carbohydrate moiety or can be an organic or inorganic compound sufficient to direct an attached molecule to a desired location.

### II. Combination Therapeutics for Treating Cancer

It has been discovered that capsaicin causes a specific cellular degradation of the inducible form of Hsp70 (also referred to as Hsp70i), along with several Hsp90 client proteins, leading to cancer cell death by apoptosis. Capsaicin causes the degradation of Hsp70/Hsp90 complexes, providing a means to overcome the increased pro-survival signaling that results from the use of many Hsp90 inhibitors.

Pharmaceutical compositions including an effective amount of a capsaicin, a synthetic capsaicin, or a derivative, analog or prodrug, or a pharmacologically active salt thereof and one or more inhibitors of Hsp90 to reduce, decrease, or inhibit the Hsp70 and Hsp90 chaperone pathways in cells compared to a control are provided. In one embodiment, the Hsp70 inhibitor selectively inhibits the inducible form of Hsp70 and not the constitutive form Hsc70.

In one embodiment, the inhibitor of Hsp90 binds to the ATP binding pocket in the N-terminal domain of Hsp90. The inhibitors of Hsp90 can be classified according to their similarity to geldanamycin (GM), radicicol (RD) or to the purine-scaffold. Geldanamycin derivatives include but are not limited to 17-AAG (17-allyl-17-desmethoxygeldanamycin); 17-DMAG (17-desmethoxy-17-N,N-dimethylaminoethylaminogeldanamycin); IPI-504 [17-allylamino-17-demethoxygeldanamycin hydroquinone hydrochloride]; and IPI-493 (17-desmethoxy-17-amino geldanamycin). Purine and purine-like analogues include, but are not limited to CNF 2024/BIIB021; MPC-3100; Debio 0932 (CUDC-305); and PU-H71. Resorcinol derivatives include, but are not limited to STA-9090 (Ganetespib); NVP-AUY922/VER52296; KW-2478; and AT13387. Other Hsp90 inhibitors include dihydroindazolone derivatives such as SNX-5422. Still other Hsp90 inhibitors include DS-2248 and XL-888. See (Jhaveri et al., Biochim Biophys Acta., 1823(3):742-755 (2012) which is incorporate herein by reference in its entirety).

A. Inhibitors of Hsp90

Heat shock proteins (Hsp) are chaperone proteins that become up-regulated in response to cellular environmental stresses, such as elevated temperature and oxygen or nutrient deprivation. Hsp chaperones facilitate proper folding and repair of other cellular proteins, referred to as “client proteins”, and also aid the refolding of misfolded proteins. The Hsp90 family is one of the most abundant families of Hsps, representing approximately 1-2% of the total protein content in non-stressed cells and 4-6% of the protein content of cells that are stressed.

The N-terminal domain of Hsp90 contains an ATP-binding site that is central to the chaperone function. The C-terminal domain of Hsp90 mediates constitutive Hsp90 dimerization. Conformational changes of Hsp90 are orchestrated with the hydrolysis of ATP.

Hsp90 is highly conserved and facilitates folding and maturation of over 200 client proteins which are involved in a broad range of critical cellular pathways and processes. In non-stressed cells Hsp90 participates in low affinity interactions to facilitate protein folding and maturation. In stressed cells Hsp90 can assist the folding of dysregulated proteins, and is known to be involved in the development and maintenance of multiple diseases.

Hsp90 maintains the conformation and stability of many oncogenic proteins, transcription factors, steroid receptors, metalloproteases and nitric oxide synthases that are essential for survival and proliferation of cancer cells (Whitesell, et al., Nature Reviews Cancer, 5, 761-772 (2005)). Thus, Hsp90 client proteins have been associated with the development and progression of cancer. Furthermore, Hsp90 is thought to contribute to maintenance of multiple neurodegenerative diseases that are associated with protein degradation and misfolding (proteinopathy), such as Alzheimer's disease, Huntingdon's disease and Parkinson's disease, through the mis-folding or stabilization of aberrant (neurotoxic) client-proteins.

Inhibition of Hsp90 function results in the misfolding of client proteins, which are subsequently ubiquitinated and degraded through proteasome-dependent pathways.

It is now accepted that at the phenotypic level, the Hsp90 pathway serves as a biochemical buffer for the numerous cancer-specific lesions that are characteristic of diverse tumors. Successful validation of Hsp90 as a target in cancer through the use of pharmacologic agents led to the development of a number of Hsp90 inhibitors which have been the subject of numerous clinical trials (reviewed in Taldone, et al., Curr. Opin. Pharmacol 8(4): 370-374 (2008)).

Hsp90 inhibitors bind to Hsp90, and induce the proteasomal degradation of Hsp90 client proteins. Although Hsp90 is highly expressed in most cells, Hsp90 inhibitors selectively kill cancer cells compared to normal cells. A number of Hsp90 inhibitors are known in the art. Most known Hsp90 inhibitors act by binding to the N-terminus of Hsp90 and disrupting the interaction between Hsp90 and heat shock factor 1 (HSF-1). However, these Hsp90 inhibitors induce an increase in expression of Hsp70 (Bagatell, et al., Clin. Cancer Res., 6(8):3312-8 (2000)).

1. Ansamycins

Drug-sensitivity of Hsp90 was established using members of the natural ansamycin family of antibiotics, which were the first Hsp90 inhibitors with antitumor activity identified. These antibiotics contain benzoquinone structures that confer selectivity for Hsp90 inhibition and can be used in the disclosed compositions and methods. Use of the ansamycins and derivatives as chemical probes has also been important in understanding the role of Hsp90 in stabilizing oncoproteins and how destabilizing Hsp90:client complexes leads to their cellular degradation, mainly through the proteasome pathway and cancer cell death. Since their discovery, these molecules have been widely explored due to their broad-spectrum antitumor activity. In some embodiments, the HSP90 inhibitor is an ansamycin.

a. Geldanamycin

Geldanamycin (Formula I) is a naturally-occurring benzoquinone ansamycin antibiotic produced by Streptomyces hygroscopicus. Geldanamycin binds with high affinity to the N-terminal ATP binding pocket of Hsp90 and induces degradation of proteins that are mutated in cancer cells preferentially over their normal cellular counterparts. However, geldanamycin has multiple drawbacks in the clinic, including hepatotoxicity, that have prevented its widespread use in cancer therapy.

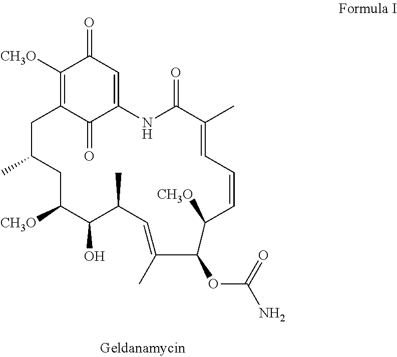

b. Tanespimycin (17-AAG)

17-allylamino-17-demethoxygeldanamycin, also known as Tanespimycin and 17-AAG (Formula II), is a less toxic and more stable analog of geldanamycin. Although binding of 17-AAG to Hsp90 is weaker than that of geldanamycin, 17-AAG displays similar antitumor effects and a better toxicity profile. 17-AAG exhibits low toxicity toward non-tumor cells and has more than 100× higher affinity for Hsp90 derived from transformed cells overexpressing HER-2 (BT474, N87, SKOV3 and SKBR3) or BT474 breast carcinoma cells with IC50 values of 5-6 nM (Kamal, et al., Nature, 425:407-410 (2003); Solit, et al., Clin Cancer Res, 8:986-993 (2002)). A 17-AAG dose of 50 mg/kg was effective in mouse xenograft studies (52). Hence, 17-AAG has been the subject of multiple phase trials for cancer treatment.

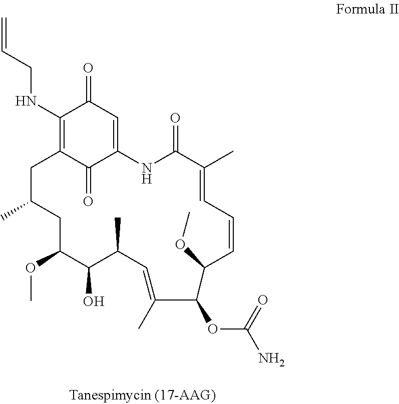 [SMILES: C=CCNC1=C2C[C@@H](C)C[C@H](OC)[C@H](O)[C@@H](C)/C=C(\C)[C@H](OC(N)=O)[C@@H](OC)/C=C\C=C(/C)C(=O)NC(=CC1=O)C2=O]

c. Alvespimycin (17-DMAG)

17-Dimethylaminoethylamino-17-demethoxygeldanamycin (also known as Alvespimycin or 17-DMAG (Formula III)) is a semi-synthetic derivative of Geldanamycin also being studied for the possibility of treating cancer. 17-DMAG is the first water-soluble analog of 17-AAG that shows promise in preclinical models, as it has excellent bioavailability, is widely distributed to tissues, and is quantitatively metabolized much less than is 17-AAG. 17-DMAG binds to the ATPase site of human Hsp90α with high affinity (Growth Inhibition of 50% (GI50)=51 nM for 17-DMAG vs. 120 nM for 17-AAG in the NCI 60-cell panel in vitro activity screen) (see Egorin, et al., Cancer Chemother. Pharmacol., 49:7-19 (2002) and Workman, et al., Curr Cancer Drug Targets, 3:297-300 (2003)).

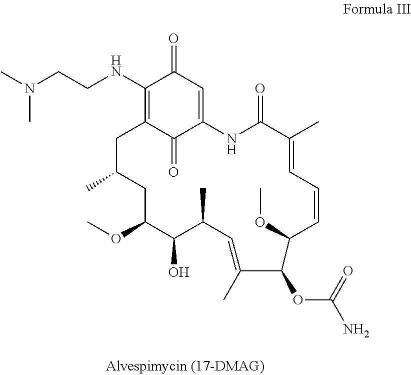 [SMILES: CO[C@H]1/C=C\C=C(/C)C(=O)NC2=CC(=O)C(NCCN(C)C)=C(C[C@@H](C)C[C@H](OC)[C@H](O)[C@@H](C)/C=C(\C)[C@@H]1OC(N)=O)C2=O]

d. Retaspimycin HCl (IPI-504)

Retaspimycin hydrochloride (also known as IPI-504 (Formula IV)) is a semi-synthetic derivative of Geldanamycin with anti-proliferative and antineoplastic activities that is being studied for the possibility of treating cancer. IPI-504 is a water-soluble analog of 17-AAG that has excellent bioavailability and has emerged as a potential replacement for 17-AAG. In the circulation, retaspimycin HCl is deprotonated and the free base hydroquinone is oxidized to 17-AAG; 17-AAG is subsequently reduced back to the hydroquinone by cellular reductase enzymes, such that the two moieties exist in a dynamic equilibrium in vivo (see Modi, et al., Breast Cancer Res., 139:107-113 (2013); Siegel, et al., Leuk. Lymphoma, 52:2308-2315 (2011)).

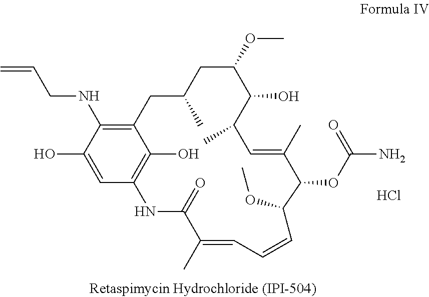 [SMILES: C=CCNc1c(O)cc2c(O)c1C[C@@H](C)C[C@H](OC)[C@H](O)[C@@H](C)/C=C(\C)[C@H](OC(N)=O)[C@@H](OC)/C=C\C=C(/C)C(=O)N2 ; Cl]

2. Ganetespib (STA-9090)

One promising antitumor agent currently in multiple clinical trials is STA-9090 (Formula V) (Synta Pharmaceuticals) (McCleese, et al., Int J Cancer 125:2792-2801(2009); Ying, et al., Mol. Cancer Ther. 11:475-484 (2012); Shimamura, et al., Clin. Cancer Res., 18, 4973-4985 (2012)). Ganetespib is synthetic non-geldanamycin inhibitor of Hsp90 that also binds the N-terminus ATP-binding domain. Preclinical data indicate that STA-9090 has a greater potency than 17-AAG with greater distribution throughout the tumor and no evidence of cardiac or liver toxicity (Ying, et al., Mol. Cancer Ther. 11:475-484 (2012); Shimamura, et al., Clin. Cancer Res., 18, 4973-4985 (2012)). Ganetespib exhibits sustained activity even with short exposure times; the 50% inhibitory concentrations (IC50) for Ganetespib against malignant mast cell lines are 10-50 times lower than that for 17-AAG (see Shimamura, et al., Clin. Cancer Res., 18, 4973-4985 (2012)). STA-9090 induces the overexpression of Hsp70.

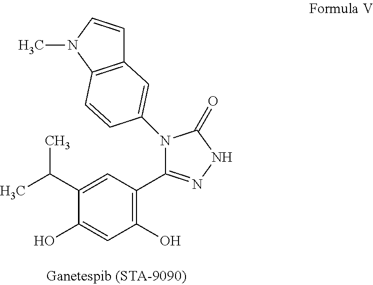 [SMILES: CC(C)c1cc(-c2n[nH]c(=O)n2-c2ccc3c(ccn3C)c2)c(O)cc1O]

3. Novobiocin

A second ATP binding site has been identified in the C-terminus of Hsp90, and compounds that interact with it, such as novobiocin, induce apoptotic cell death of cancer cells. Chemical optimization of novobiocin has led to a significant improvement in its efficacy in killing cancer cells.

4. Other Inhibitors of Hsp90

In addition to the geldanamycin derivatives, a series of purine scaffold inhibitors have been developed and have entered clinical trials. Many different Hsp90 inhibitors are known in the art, including C-11, SNX-2112, SNX-5542, NVP-AUY922, NVP-BEP800, CCT018159, VER-49009, PU3, BIIB021, herbimycin, derrubone, gedunin, celastrol (tripterine), (−)-epigallocatechin-3-gallate ((−)-EGCG), KW-2478, radicicol, radicicol oxime derivatives, radamide, radester, radanamycin, AT13387, debio0932, XL888 and pochonin A-F (see Hao, et al., Oncology Reports, 23:1483-92 (2010)). Diverse chemical scaffolds that have been developed as Hsp90 inhibitors are known in the art, including resorcinols, pyrimidines, aminopyrimidine, azoles and other chemotypes.

B. Capsaicin, Derivatives, and Analogs Thereof

The evolutionarily-conserved Hsp70 chaperones consist of an N-terminal ATPase domain of 45 kDa and a C-terminal substrate-binding domain of 25 kDa (Mayer, et al., Cell Mol Life Sci, 62:670-684 (2005)). Genetic and biochemical evidence clearly demonstrate that ATP hydrolysis is essential for Hsp70. The hydrolysis of ATP triggers the closing of the substrate binding cavity and the locking-in of associated substrates. In turn, substrates stimulate the hydrolysis of ATP by several-fold but further stimulation of the Hsp70 ATPase activity is provided by interaction with the J-domain co-chaperones (Hsp40). In certain cases, Hsp40 and substrate stimulate the rate of ATP hydrolysis by more than 1000-fold.

Hsp70 acts as a natural inhibitor of several stress-kinases at the beginning of cell death, and protects cells from apoptosis by binding and modulating the activity of various pro- and anti-apoptotic proteins at the transcriptional and posttranslational level. Hence, Hsp70 is renowned as an anti-apoptotic factor. The two major cytoplasmic isoforms of Hsp70 are HSC70 and Hsp72. Generally, HSC70 is abundantly and ubiquitously expressed in non-tumor tissues, whereas Hsp72 is present at relatively low levels in the absence of stress (Daugaard et al., Cancer Res., 67:2559-2567 (2007)). However, under stress, the expression of inducible Hsp72 increases considerably via heat-shock factor 1 (HSF1) transcription factor activation. This differential expression pattern is commonly lost in cancer and a large body of evidence supports the role of the heat shock proteins in maintaining the cancer phenotype.

Inhibition of the Hsp70 chaperone pathway simultaneously disrupts several critical cancer survival pathways, supporting the idea of targeting Hsp70 as a potential approach for cancer therapeutics (Leu, et al., Mol. Cancer Res., 9:936-947 (2011); Nylandsted, et al., Proc. Natl. Acad. Sci. USA, 97:7871-7876 (2000)).

Furthermore, the inhibition of Hsp70 can influence other pro-cancer survival factors, such as Hsp90. Hsp70 chaperones function as co-chaperones for Hsp90 to maintain the stability and activities of the Hsp90 client proteins, many of which are known to be associated with the development and progression of cancer (Whitesell, et al., Nature Reviews Cancer, 5, 761-772 (2005)).

It has been discovered that disruption of Hsp70 by capsaicin leads to cancer cell death by apoptosis. It is believed that capsaicin inhibits the Hsp70 chaperone pathway, leading to inhibition of Hsp70-associated factors such as the Hsp70/Hsp90 complex. Hence, the mechanism for cytotoxicity of cancer cells is thought to involve the inhibition of Hsp70/Hsp90 chaperone complex function.

The combination of capsaicin and Hsp90 inhibitors abrogate the protective mechanism provided by Hsp70 thereby increasing its cytotoxic effect.

1. Capsaicin

Capsaicin is a member of the capsaicinoid group of compounds that are characterized as containing a 3-hydroxy-4-methoxy-benzylamide (vanilloid ring) pharmacophore and a hydrophobic alkyl side chain. Capsaicinoids are the compounds responsible for the pungency of pepper fruits and their products.

Capsaicin was first isolated in 1876 and the empirical structure was first determined in 1919 as being C₁₈H₂₇NO₃ (8-Methyl-N-vanillyl-trans-6-nonenamide). Capsaicin has a molecular weight of 305 daltons and contains a vanilloid ring pharmacophore with a hydrophobic alkyl side chain of 11 carbons, according to Formula VI. The double bond structure within the hydrophobic alkyl side chain prevents internal rotation and the molecule displays cis/trans isomerism. However, the cis isomer is a less-stable arrangement and so capsaicin is naturally present as the trans isomer.

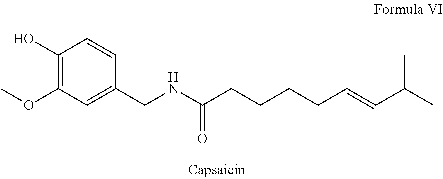 [SMILES: COc1cc(CNC(=O)CCCC/C=C/C(C)C)ccc1O]

Capsaicin's volatility is very low and it is completely odorless. Purified capsaicin is a waxy, colorless substance at room temperature and is insoluble in cold water, but freely soluble in alcohol, fats and oils. The extended hydrocarbon tail enables incorporation of capsaicin into lipid-rich cell membranes and capsaicin is known to be effectively absorbed topically from the skin and mucosa. The pharmacokinetic half-life of capsaicin was found to be approximately 24 hours.

Capsaicin is FDA approved as a topical drug for chronic pain management. However, it has not been tested in a clinical trial as an anticancer agent. Numerous studies indicate that capsaicin has antitumor activity in vitro and in vivo. Capsaicin was reported to bind the transient receptor potential vanilloid (TRPV1) (Caterina, et al., Nature, 389:816-824 (1997)), which mediates noxious stimuli in sensory neurons. It was shown to be safe in animal studies (Park, et al., Anticancer Res., 18:4201-4205 (1998)). Reports show that capsaicin induced apoptotic cell death in a large panel of cancer cell lines, including androgen receptor (AR)-positive (LNCaP) and -negative (PC-3, DU145) prostate carcinoma cell lines (Mori, et al., Cancer Res., 66:3222-3229 (2006); Sanchez, et al., Apoptosis, 11:89-99 (2006)) estrogen receptor (ER)-positive (MCF7, T47D and BT-474), ER-negative (SKBR-3) and triple-negative (MDA-MB231) breast carcinomas (Thoennissen, et al., Oncogene 29, 285-296 (2010)), glioblastoma (A172) (Lee, et al., Cancer Letts, 161:121-130 (2000)), hepatoma HepG2 cells (Huang, et al., Anticancer Res 29:165-174 (2009)), and pancreatic cancer cells through the phosphoinositide 3-kinase/Akt pathway (Zhang, et al., Apoptosis, 13:1465-147837(2008); Zhang, et al., Oncol. Lett. 5:43-48 (2013)).

Other reports indicate that capsaicin induced bladder carcinoma cell death by inhibiting cyclin dependent kinase (CDK4) (Chen, et al., Int. J. Urol., 19:662-668 (2012)) and through reactive oxygen species production and mitochondrial depolarization (Yang, et al., Urology 75:735-741 (2010)). Capsaicin displayed anti-proliferative activity against human small cell lung cancer via the E2F pathway (Brown, et al., PLoS ONE 5:e1024341 (2010)), repressed the transcriptional activity of β-catenin in human colorectal cancer cells (Lee, et al., J. Nutr. Biochem., 23:646-655 (2012)), blocked matrix metalloproteinase-9 expression and inhibited FAK/Akt and Raf/ERK signaling (Hwang, et al, Mol. Nutr. Food Res., 55:594-60543 (2011)). Capsaicin sensitized malignant glioma cells to TRAIL-mediated apoptosis via DR5 up-regulation and survivin down regulation (Kim, et al., Carcinogenesis 31, 367-375 (2010)).

The anti-tumor activity of capsaicin was demonstrated in mouse xenograft models (Mori, et al., Cancer Res., 66:3222-3229 (2006); Sanchez, et al., Apoptosis, 11:89-99 (2006)) in which 5 mg/kg capsaicin was given to animals by gavage, three days per week for 2-4 weeks. No toxicity symptoms were observed and the animals showed high tolerability to capsaicin.

It is believed that the mechanism by which capsaicin induces apoptosis in cancer cells is independent of the TRPV1 receptor. Neither capsazepine, a powerful vanilloid receptor antagonist, nor intracellular Ca²⁺ chelators significantly inhibited the capsaicin-induced apoptosis (Lee, et al., Cancer Letts., 161:121-130 (2000); Athanasiou, et al., Biochem. Biophys. Res. Commun. 354:50-55(2007)). Capsaicin blocked the migration of breast cancer cell lines in vitro and significantly slowed the growth of prostate PC-3, breast MDA-MB231 and bladder T24 (Yang, et al., Urology 75:735-741 (2010); Brown, et al., PLoS ONE 5:e1024341 (2010)) orthotopic tumors in mouse xenograft models. It also inhibited benzo(a)pyrene-induced lung carcinogenesis in mouse model (Anandakumar, et al., Inflammation research: official journal of the European Histamine Research Society, 61:1169-1175 (2012)).

Recently, a combination of capsaicin with drugs targeting tumor metabolism such as alpha-lypoic acid and hydroxycitrate, caused tumor regression in three animal models: lung cancer, bladder cancer and melanoma (Schwartz, et al., Can. Urol. Assoc. J., 4:E9-Ell (2010)). Capsaicin induced down-regulation of prostate-specific antigen (PSA) and AR in LNCaP cells (Schwartz, et al., Can. Urol. Assoc. J., 4:E9-Ell (2010)), and reduced ERK activation and HER-2 expression in breast cancer cell lines. In humans, a case report of a patient with prostate cancer indicated that capsaicin may slow the doubling time of PSA (Jankovic, et al., J. Clin. Oncol., 15:2974-2980 (1997)), and capsaicin cream can effectively control post-surgical pain in cancer patients (Ellison, et al., J. Clin. Oncol., 15, 2974-2980 (1997)).

2. Sources of Capsaicin

a. Plants

Capsaicinoids are naturally occurring compounds which are the active component of chili peppers and are renowned for their use in culinary applications worldwide. Capsaicinoids are produced within fruit of plants belonging to the Capsicum genus of the family Solanaceae. The fruits of “spicy” pepper plants, such as those commonly known as jalapenos or habaneros are an abundant natural source of capsaicin. The capsaicin is located between the seeds and the rib of the pepper fruit and is retained within pepper fruits that are dried and/or ground. Examples of Capsicum species renowned for high capsaicinoid content include C. annum (Oleoresin red pepper), C. frutescens (Jalapeno pepper) and C. chinense (Habanero pepper), which were found to contain 0.22-20 mg total capsaicinoids/g of dry weight. Capsaicin is the most abundant capsaicinoid within pepper plants, accounting for ˜71% of the total capsaicinoids in most of the pungent varieties (see de Lourdes Reyes-Escogido et al, Molecules, 16:1253-1270 (2011)). Capsaicin biosynthesis involves condensation of vanillylamine and 8-methyl nonenoic acid, brought about by the enzyme capsaicin synthase (CS).

b. Synthetic Sources

Purified capsaicin is commercially available (Sigma Aldrich # M2808, CAS#404-86-4). Capsaicin and its analogs are produced industrially using chlorinated fatty acids and amines at temperatures between 140 and 170° C. under moderate pressure (see Kaga, et al., J. Org. Chem., 54:3477-3478, 1989 and Kaga, et al., Tetrahedron 1996, 52, 8451-8470). However, application of large-scale chemical synthesis of capsaicin is limited by the toxicity of the required reagents, a disadvantage which makes enzymatic synthesis an appealing alternative to traditional chemical synthesis. Hence, several methods are known for the in vitro chemical synthesis of capsaicinoids from substrate molecules using enzyme-catalyzed reactions (see, for example, Kobata, et al., Biotechnol. Lett., 21, 547-550 (1999)).

The enzymatic formation of capsaicin in vitro has been demonstrated using cells and tissues from the plant Capsicum annum, grown in liquid media (see Johnson, et al., Plant Sci. 70:223-229 (1990)). Cells and placental tissues from fruits, grown in vitro immobilized in calcium alginate produced capsaicin in the medium. Greater potentiality for capsaicin synthesis was observed in immobilized placental tissue than immobilized cells. A maximum yield of 2,400 μg capsaicin/g immobilized placenta was observed after 30 days of culture.

3. Analogs of Capsaicin

a. Natural Capsaicin Analogs

i. Capsaicinoids

In some embodiments, the capsaicin is a capsaicinoid analog of capsaicin. Capsaicinoid analogs of capsaicin are known in the art. See, for example, Reilly and Yost, Drug Metab. Disp., 33:550-536 (2005) which is incorporated by reference herein in its entirety. Reilly and Yost describe five naturally occurring capsaicinoid analogs of capsaicin. These compounds possess the same 3-hydroxy-4-methoxy-benzylamide (vanilloid ring) pharmacophore, but have differences in the hydrophobic alkyl side chain moiety, such as saturation of C15-16 (the ω-2,3 position), deletion of a methyl group at C17 (loss of the tertiary carbon), and changes in the length of the hydrocarbon chain. Naturally occurring capsaicinoid analogues of capsaicin include, but are not limited to, homocapsaicin, nordihydrocapsaicin, dihydrocapsaicin, homodihydrocapsaicin, n-vanillyloctanamide, nonivamide and n-vanillyldecanamide, as in Formula VII.

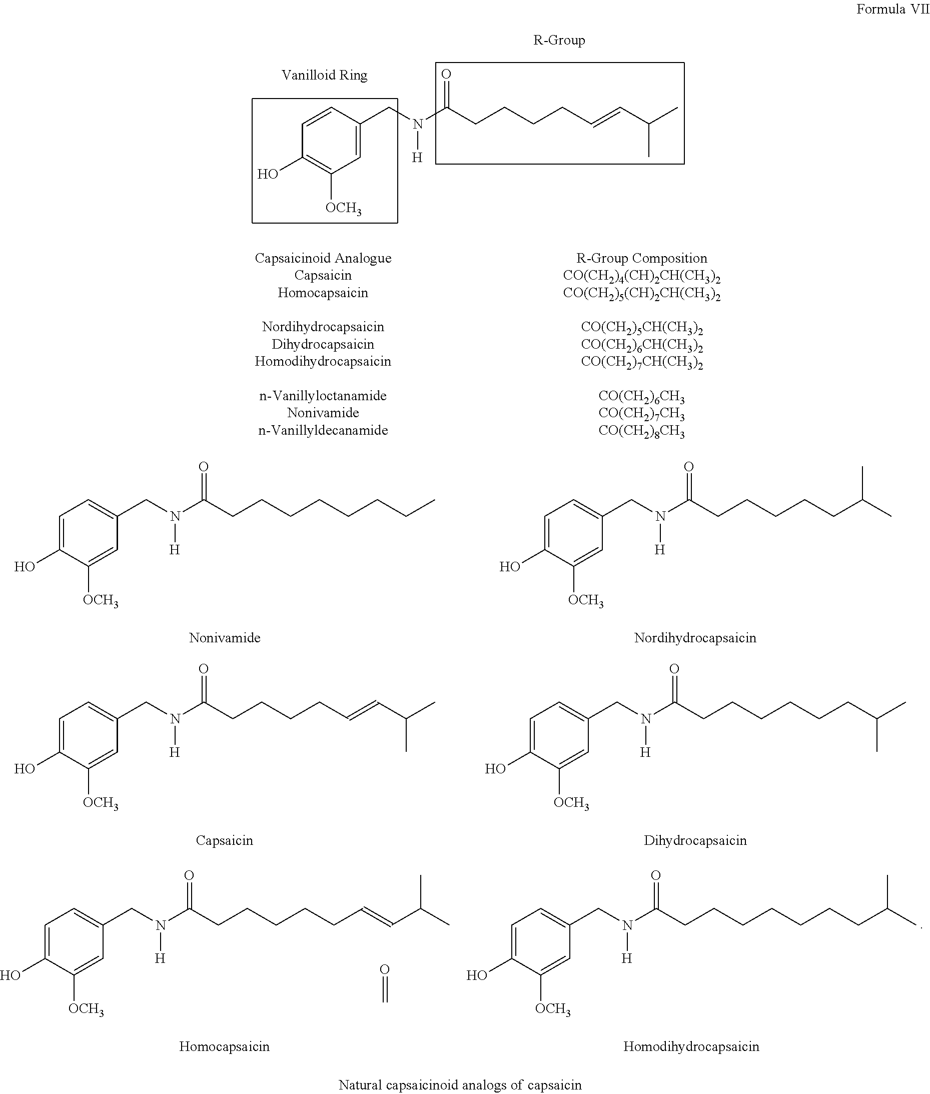

ii. Capsinoids

In some embodiments the capsaicin is a capsinoid analogue of capsaicin. The capsinoid group of compounds, including capsiate, dihydrocapsiate and nordihydrocapsoate, are structurally similar to the capsaicinoids, but have a different center linkage, which is an amide moiety in the capsaicinoids and an ester moiety in capsinoids, as in Formula VIII. Capsinoids are isolated from a few varieties of non-pungent red pepper plants, such as the CH19 sweet cultivar. The bio-potency of capsiate is similar to capsaicin, however capsinoids to not exhibit pungency or sensory irritation.

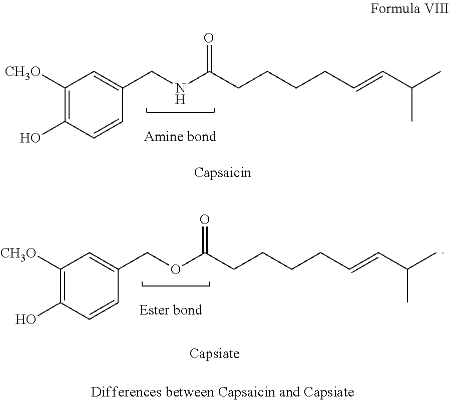

b. Synthetic Capsaicin Analogs

In some embodiments the capsaicin is a synthetic capsaicinoid or capsinoid analogue of capsaicin. Multiple synthetic (non-naturally occurring) analogues of capsaicin are known in the art (see, for example, Satoh, et al., Biochim. Biophys. Acta., 1273:21-30 (1996) which is incorporated by reference herein in its entirety). Pungent and non-pungent analogues can be synthesized using different acyl chain lengths and/or chemical substitutions in the aromatic ring. Specifically, both capsaicinoids and capsinoids can be synthetically modified through modification of the substitution pattern and/or the number of methoxy groups on the benzene ring, which may be superimposable on the quinone ring of ubiquinone. In addition, the capsaicin may be modified through alteration of the position of dipolar amide bond unit in the molecule and/or other chemical modifications of this unit. Examples of chemical modifications are given in Formula IX.

In some embodiments the synthetic capsaicin analog is designed such that chemical modification enhances the efficacy of the capsaicin as an inhibitor of the Hsp70 pathway.

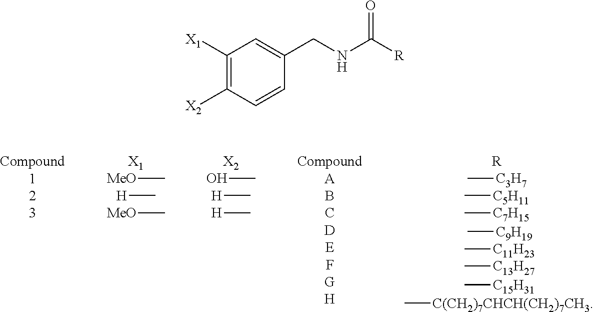

C. Formulations

Pharmaceutical compositions including an effective amount of a capsaicin, a synthetic capsaicin, or a derivative, analog or prodrug, or a pharmacologically active salt thereof and one or more inhibitors of Hsp90 and Hsp70 to reduce, decrease, or inhibit the Hsp70 and Hsp90 chaperone pathways in cells compared to a control are disclosed. The disclosed compositions can be formulated as pharmaceutical compositions. In a preferred embodiment, the Hsp70 inhibitor selectively inhibits induced Hsp70 and does not substantially inhibit Hsc70, the constitutive form of Hsp70.

Pharmaceutical compositions may be for administration by oral, parenteral (intramuscular, intraperitoneal, intravenous (IV) or subcutaneous injection), transdermal (either passively or using iontophoresis or electroporation), transmucosal (nasal, vaginal, rectal, or sublingual) routes of administration or using bioerodible inserts and can be formulated in unit dosage forms appropriate for each route of administration.

In some embodiments the naturally occurring capsaicin, synthetic capsaicin, or derivative, analog or prodrug, or pharmacologically active salt thereof is in a delivery vehicle to facilitate administration.

1. Parenteral Administration

In one embodiment, the compositions are administered in an aqueous solution, by parenteral injection. The formulation may also be in the form of a suspension or emulsion. In general, pharmaceutical compositions are provided including effective amounts of capsaicin, or a derivative, analog or prodrug, or a pharmacologically active salt thereof and optionally include pharmaceutically acceptable diluents, preservatives, solubilizers, emulsifiers, adjuvants and/or carriers. Such compositions include diluents such as sterile water, buffered saline of various buffer content (e.g., Tris-HCl, acetate, phosphate), pH and ionic strength; and optionally, additives such as detergents and solubilizing agents (e.g., TWEEN®20, TWEEN®80, Polysorbate 80), anti-oxidants (e.g., ascorbic acid, sodium metabisulfite), and preservatives (e.g., Thimersol, benzyl alcohol) and bulking substances (e.g., lactose, mannitol). Examples of non-aqueous solvents or vehicles are propylene glycol, polyethylene glycol, vegetable oils, such as olive oil and corn oil, gelatin, and injectable organic esters such as ethyl oleate. The formulations may be lyophilized and redissolved/resuspended immediately before use. The formulation may be sterilized by, for example, by filtration through a bacteria retaining filter, by incorporating sterilizing agents into the compositions, by irradiating the compositions, or by heating the compositions.

2. Enteral Administration

The compositions can be formulated for oral delivery.

a. Additives for Oral Administration

Oral solid dosage forms are described generally in Remington's Pharmaceutical Sciences, 18th Ed. 1990 (Mack Publishing Co. Easton Pa. 18042) at Chapter 89. Solid dosage forms include tablets, capsules, pills, troches or lozenges, cachets, pellets, powders, or granules or incorporation of the material into particulate preparations of polymeric compounds such as polylactic acid, polyglycolic acid, etc. or into liposomes. Such compositions may influence the physical state, stability, rate of in vivo release, and rate of in vivo clearance of the present active compounds and derivatives. See, e.g., Remington's Pharmaceutical Sciences, 18th Ed. (1990, Mack Publishing Co., Easton, Pa. 18042) pages 1435-1712, which are herein incorporated by reference. The compositions may be prepared in liquid form, or may be in dried powder (e.g., lyophilized) form. Liposomal or proteinoid encapsulation may be used to formulate the compositions (as, for example, proteinoid microspheres reported in U.S. Pat. No. 4,925,673). Liposomal encapsulation may be used and the liposomes may be derivatized with various polymers (e.g., U.S. Pat. No. 5,013,556). See also Marshall, K. In: Modern Pharmaceutics Edited by G. S. Banker and C. T. Rhodes Chapter 10, 1979. In general, the formulation will include the compound (or chemically modified forms thereof) and inert ingredients which protect the compound in the stomach environment, and release of the biologically active material in the intestine.

Another embodiment provides liquid dosage forms for oral administration, including pharmaceutically acceptable emulsions, solutions, suspensions, and syrups, which may contain other components including inert diluents; adjuvants such as wetting agents, emulsifying and suspending agents; and sweetening, flavoring, and perfuming agents.

Controlled release oral formulations may be desirable. Capsaicin, or a derivative, analog or prodrug, or a pharmacologically active salt thereof can be incorporated into an inert matrix which permits release by either diffusion or leaching mechanisms, e.g., gums. Slowly degenerating matrices may also be incorporated into the formulation. Another form of a controlled release is based on the Oros therapeutic system (Alza Corp.), i.e., the drug is enclosed in a semipermeable membrane which allows water to enter and push drug out through a single small opening due to osmotic effects. For oral formulations, the location of release may be the stomach, the small intestine (the duodenum, the jejunum, or the ileum), or the large intestine. Preferably, the release will avoid the deleterious effects of the stomach environment, either by protection of the active agent (or derivative) or by release of the active agent (or derivative) beyond the stomach environment, such as in the intestine. To ensure full gastric resistance a coating impermeable to at least pH 5.0 is essential. Examples of the more common inert ingredients that are used as enteric coatings are cellulose acetate trimellitate (CAT), hydroxypropylmethylcellulose phthalate (HPMCP), HPMCP 50, HPMCP 55, polyvinyl acetate phthalate (PVAP), Eudragit L30D, Aquateric, cellulose acetate phthalate (CAP), Eudragit L, Eudragit S, and Shellac. These coatings may be used as mixed films.

b. Chemically Modified Forms for Oral Dosage

Capsaicin, or a derivative, analog or prodrug, thereof may be chemically modified so that oral delivery of the derivative is efficacious. Generally, the chemical modification contemplated is the attachment of at least one moiety to the component molecule itself, where said moiety permits (a) inhibition of proteolysis; and (b) uptake into the blood stream from the stomach or intestine. Also desired is the increase in overall stability of the component or components and increase in circulation time in the body. PEGylation is a preferred chemical modification for pharmaceutical usage. Other moieties that may be used include: propylene glycol, copolymers of ethylene glycol and propylene glycol, carboxymethyl cellulose, dextran, polyvinyl alcohol, polyvinyl pyrrolidone, polyproline, poly-1,3-dioxolane and poly-1,3,6-tioxocane (see, e.g., Abuchowski and Davis (1981) “Soluble Polymer-Enzyme Adducts,” in Enzymes as Drugs. Hocenberg and Roberts, eds. (Wiley-Interscience: New York, N.Y.) pp. 367-383; and Newmark, et al., (1982) J. Appl. Biochem. 4:185-189).

3. Controlled Delivery Polymeric Matrices

Controlled release polymeric devices can be made for long term release systemically following implantation of a polymeric device (rod, cylinder, film, disk) or injection (microparticles). The matrix can be in the form of microparticles such as microspheres, where peptides are dispersed within a solid polymeric matrix or microcapsules, where the core is of a different material than the polymeric shell, and the peptide is dispersed or suspended in the core, which may be liquid or solid in nature. Unless specifically defined herein, microparticles, microspheres, and microcapsules are used interchangeably. Alternatively, the polymer may be cast as a thin slab or film, ranging from nanometers to four centimeters, a powder produced by grinding or other standard techniques, or even a gel such as a hydrogel.

Either non-biodegradable or biodegradable matrices can be used for delivery of capsaicin, although biodegradable matrices are preferred. These may be natural or synthetic polymers, although synthetic polymers are preferred due to the better characterization of degradation and release profiles. The polymer is selected based on the period over which release is desired. In some cases linear release may be most useful, although in others a pulse release or “bulk release” may provide more effective results. The polymer may be in the form of a hydrogel (typically in absorbing up to about 90% by weight of water), and can optionally be cross-linked with multivalent ions or polymers.

The matrices can be formed by solvent evaporation; spray drying, solvent extraction and other methods known to those skilled in the art. Bioerodible microspheres can be prepared using any of the methods developed for making microspheres for drug delivery, for example, as described by Mathiowitz and Langer, J. Controlled Release 5, 13-22 (1987); Mathiowitz, et al., Reactive Polymers 6, 275-283 (1987); and Mathiowitz, et al., J. Appl. Polymer Sci. 35, 755-774 (1988).

The devices can be formulated for local release to treat the area that is subject to a disease, which will typically deliver a dosage that is much less than the dosage for treatment of an entire body or systemic delivery. These can be implanted or injected subcutaneously, into the muscle, fat, or swallowed.

4. Topical Administration

Topical administration of capsaicins may be desirable. In some embodiments capsaicin, or a derivative, analog or prodrug, or a pharmacologically active salt thereof can be incorporated into an inert matrix to be administered in the form of a suppository or pessary, or may be applied topically in the form of a lotion, solution, cream, ointment or dusting powder. Capsaicins may also be transdermally administered, for example, by the use of a skin patch or other intra-dermal devices. They may also be administered by the ocular route. For application topically to the skin, the capsaicins can be formulated as a suitable ointment containing the active compound suspended or dissolved in, for example, a mixture with one or more of the following: mineral oil, liquid petrolatum, white petrolatum, propylene glycol, polyoxyethylene polyoxypropylene compound, emulsifying wax and water. Alternatively, they can be formulated as a suitable lotion or cream, suspended or dissolved in, for example, a mixture of one or more of the following: mineral oil, sorbitan monostearate, a polyethylene glycol, liquid paraffin, polysorbate 60, cetyl esters wax, cetearyl alcohol, 2-octyldodecanol, benzyl alcohol and water.

In some embodiments, the topical administration is in the mouth. Formulations suitable for topical administration in the mouth include lozenges including the capsaicin in a flavored basis, usually sucrose and acacia or tragacanth; pastilles including the capsaicin in an inert basis such as gelatin and glycerin, or sucrose and acacia; and mouthwashes including the capsaicin in a suitable liquid carrier.

D. Targeting Moieties

In some embodiments, the composition includes a targeting signal, a protein transduction domain or a combination thereof. The targeting moiety can be attached or linked directly or indirectly to capsaicin, or a derivative, analog or prodrug thereof. For example, in preferred embodiments, the targeting moiety is attached or linked to a capsaicin delivery vehicle such as a nanoparticle or a microparticle.

The targeting signal or sequence can be specific for a host, tissue, organ, cell, organelle, non-nuclear organelle, or cellular compartment. Moreover, the compositions disclosed here can be targeted to other specific intercellular regions, compartments, or cell types.

In one embodiment, the targeting signal binds to its ligand or receptor which is located on the surface of a target cell such as to bring the capsaicin and cell membranes sufficiently close to each other to allow penetration of the capsaicin into the cell. Additional embodiments of the present disclosure are directed to specifically delivering capsaicin, or a derivative, analog or prodrug, or a pharmacologically active salt thereof to specific tissue or cell types with undesirable Hsp90 or Hsp 70 activity. In a preferred embodiment, the targeting molecule is selected from the group consisting of an antibody or antigen binding fragment thereof, an antibody domain, an antigen, a T-cell receptor, a cell surface receptor, a cell surface adhesion molecule, a major histocompatibility locus protein, a viral envelope protein and a peptide selected by phage display that binds specifically to a defined cell.

Ligands can be attached to polymeric particles indirectly though adaptor elements which interact with the polymeric particle. Adaptor elements may be attached to polymeric particles in at least two ways. The first is during the preparation of the micro- and nanoparticles, for example, by incorporation of stabilizers with functional chemical groups during emulsion preparation of microparticles. For example, adaptor elements, such as fatty acids, hydrophobic or amphiphilic peptides and polypeptides can be inserted into the particles during emulsion preparation. In a second embodiment, adaptor elements may be amphiphilic molecules such as fatty acids or lipids which may be passively adsorbed and adhered to the particle surface, thereby introducing functional end groups for tethering to ligands. Adaptor elements may associate with micro- and nanoparticles through a variety of interactions including, but not limited to, hydrophobic interactions, electrostatic interactions and covalent coupling.

Exemplary targeting signals include an antibody or antigen binding fragment thereof specific for a receptor expressed at the surface of a target cell or other specific antigens, such as cancer antigens. Representative receptors include but are not limited to growth factors receptors, such as epidermal growth factor receptor (EGFR; HER1; c-erbB2 (HER2); c-erbB3 (HER3); c-erbB4 (HER4); insulin receptor; insulin-like growth factor receptor 1 (IGF-1R); insulin-like growth factor receptor 2/Mannose-6-phosphate receptor (IGF-II R/M-6-P receptor); insulin receptor related kinase (IRRK); platelet-derived growth factor receptor (PDGFR); colony-stimulating factor-1receptor (CSF-1R) (c-Fms); steel receptor (c-Kit); F1k2/F1t3; fibroblast growth factor receptor 1 (Flg/Cek1); fibroblast growth factor receptor 2 (Bek/Cek3/K-Sam); Fibroblast growth factor receptor 3; Fibroblast growth factor receptor 4; nerve growth factor receptor (NGFR) (TrkA); BDNF receptor (TrkB); NT-3-receptor (TrkC); vascular endothelial growth factor receptor 1 (F1t1); vascular endothelial growth factor receptor 2/Flk1/KDR; hepatocyte growth factor receptor (HGF-R/Met); Eph; Eck; Eek; Cek4/Mek4/HEK; Cek5; Elk/Cek6; Cek7; Sek/Cek8; Cek9; Cek10; HEK11; 9 Ror1; Ror2; Ret; Ax1; RYK; DDR; and Tie.

In some embodiments, the targeting signal is or includes a protein transduction domain (PTD), also known as cell penetrating peptides (CPPS). PTDs are known in the art, and include but are not limited to small regions of proteins that are able to cross a cell membrane in a receptor-independent mechanism (Kabouridis, P., Trends in Biotechnology (11):498-503 (2003)). The two most commonly employed PTDs are derived from TAT (Frankel and Pabo, Cell, December 23; 55(6):1189-93 (1988)) protein of HIV and Antennapedia transcription factor from Drosophila, whose PTD is known as Penetratin (Derossi et al., J. Biol. Chem. 269(14):10444-50 (1994)).

### III. Methods of Treatment

Methods of inhibiting the Hsp70 and Hsp90 chaperone pathways including contacting one or more cells expressing the Hsp70/Hsp90 complex with an effective amount of a capsaicin, a synthetic capsaicin, or a derivative, analog or prodrug, or a pharmacologically active salt thereof in combination with one or more inhibitors of Hsp90 to decrease or inhibit the Hsp70 and Hsp90 chaperone pathways in the cells compared to control cells are provided.

The term “combination” or “combined” is used to refer to either concomitant, simultaneous, or sequential administration of two or more agents. Therefore, the combinations can be administered either concomitantly (e.g., as an admixture), separately but simultaneously (e.g., via separate intravenous lines into the same subject), or sequentially (e.g., one of the compounds or agents is given first followed by the second). Therefore, the combination therapy can include co-administration of the Hsp90 inhibitor and capsaicin, a synthetic capsaicin, or a derivative, analog or prodrug, or a pharmacologically active salt thereof separately in two different formulation, or together in the same formulation (i.e., a single pharmaceutical composition including both active agents). If the two agents are administered in separate formulations, co-administration can include the simultaneous and/or sequential administration of the two agents. An appropriate time course for sequential administration can be chosen by the physician, according to such factors such as the nature of a patient's illness, and the patient's condition. In certain embodiments, sequential administration includes the co-administration of the two agents within a period of hours, days, or weeks of one another. In some embodiments the inhibitor of Hsp90 is administered first, followed by the capsaicin. In some embodiments the capsaicin is administered first, followed by the inhibitor of Hsp90.

The capsaicins and inhibitors of Hsp90 can be administered locally or systemically to the subject, or coated or incorporated onto, or into a device.

A. Methods of Treating Cancer Using Combination Therapy

Methods of treating cancer including administering to a subject with cancer an effective amount of a capsaicin, a synthetic capsaicin, or a derivative, analog or prodrug, or a pharmacologically active salt thereof in combination with one or more inhibitors of the Hsp90 pathway to reduce or inhibit one or more symptoms of the cancer are provided. In a preferred embodiment, the combination of Hsp90 and Hsp70 inhibitors are contacted to a cancer cell or tumor cell and inhibit or reduce cellular proliferation or induce or promote apoptosis in the cell. Thus, one method provides a method of killing cancer cells or tumor cells by contacting the cancer or tumor cells with the disclosed compositions. The capsaicins and inhibitors of Hsp90 disclosed herein can be used in combination to provide enhanced antitumor activity as compared to the use of either agent alone.

Hsp90 inhibitors have performed poorly in the clinic as antitumor therapeutic agents. Specifically, it is well established that expression of the major inducible Hsp70 isoform, HSP72, is increased following treatment with Hsp90 inhibitor 17-AAG. Thus, the inhibition of Hsp70 molecular chaperones is of interest when considering modulation of Hsp90.

Silencing of Hsp70 genes (Hsp72 and HSC70) in human colon cancer HCT116 cells and human ovarian cancer A2780 cells significantly increased the apoptotic effects of the Hsp90 inhibitor 17-AAG. The observed increased apoptosis was specific to tumor cells (Powers, et al., Cancer Cell, 14:250-262 (2008)). Treatment of myeloma cells with 17-AAG resulted in increased Hsp72, however combinatorial treatment with both 17-AAG and inhibitors of Hsp70 blocked this increase and resulted in a decrease in proliferation compared with treatment using 17-AAG alone (Davenport, et al., Leukemia, 24:1804-1807 (2010)).

Constitutively elevated Hsp70 expression is a characteristic of many tumor cells and contributes to their survival. Specifically, Hsp70 chaperones protect cells from exposure to noxious stimuli which would otherwise cause lethal molecular damage and induce apoptosis. For example, overexpression of Hsp70 has been shown to be correlated with poor prognosis in breast cancer, endometrial cancer, uterine cervical cancer, and transitional cell carcinoma of the bladder. This is consistent with the Hsp70 associations with poor differentiation, lymph node metastasis, increased cell proliferation, block of apoptosis, and higher clinical stage, which are markers of poor clinical outcome. (Ciocca, et al., J. Natl. Cancer Inst., 85(7):570-4 (1993) and Barnes, et al., Cell Stress Chaperones, 6(4):316-25. (2001)).

Hence, inhibitors of Hsp70 represent potential therapeutics to enhance the efficacy of Hsp90 inhibitors in combination therapies to treat cancer. Small molecule inhibitors of Hsp70 have been shown to be cytotoxic in multiple human tumor cell lines, including U2OS, BX-U2OS (melanoma) cells, MiaPaCa, MDA468, MDA231, SKBR3 and MCF7 (breast cancer) cells, as well as Panc1 and CaPan1/CaPan2 (pancreas carcinoma and adenocarcinoma) cells, and impaired tumor development in a mouse model of human Burkitt's lymphoma (Leu, et al., Mol. Cell, 36:15-27 (2009)).

1. Cancers to be Treated

The compositions and methods described can be used to treat multiple types of cancer. Cytotoxic activity of capsaicin has been observed in cancer cell lines in vitro. Therefore, cancers that can be treated using the compositions and methods described herein include sarcomas, lymphomas, leukemias, carcinomas, blastomas, and germ cell tumors.

A representative but non-limiting list of cancers that the disclosed compositions and methods can be used to treat include lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, colon cancer, cervical cancer, cervical carcinoma, breast cancer including triple-negative breast cancer, epithelial cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer including hormone-refractory prostate cancer and pancreatic cancer.

2. Effective Amounts/Dosages

As used herein the term “effective amount” or “therapeutically effective amount” means a dosage sufficient to treat, inhibit, or alleviate one or more symptoms of the disorder being treated or to otherwise provide a desired pharmacologic and/or physiologic effect. Pharmaceutical compositions including an effective amount of a capsaicin, a synthetic capsaicin, or a derivative, analog or prodrug, or a pharmacologically active salt thereof and one or more inhibitors of Hsp90 to reduce, decrease, or inhibit the Hsp70 and Hsp90 chaperone pathways in cells compared to a control are provided.

In preferred embodiments, capsaicin, or a derivative, analog or prodrug, thereof reduces or inhibits the pro-survival (anti-apoptotic) pathways. Capsaicin inhibited formation of the Hsp90/Hsp70 complex by degradation of Hsp70 and prevented folding of Hsp90 client proteins. As discussed above, over-expression of the apoptosis inhibitor proteins Hsp70 and Hsp27 is associated with some Hsp90 inhibitors including 17-AAG and is thought to reduce efficacy of the inhibitors in some uses of Hsp90 inhibitors, for example, the treatment of cancer. Together these data indicate that capsaicin can be used to prevent formation and maintenance of the cancer phenotype both alone and by enhancing the pharmaceutical efficacy of Hsp90 inhibitors such as 17-AAG. Therefore, in some embodiments, the combined use of capsaicin, or a derivative, analog or prodrug thereof in combination with an inhibitor of Hsp90 enhances the half maximal inhibitory concentration (IC50) of one or both compounds relative to each compound used separately.

In some embodiments, capsaicin, or a derivative, analog or prodrug thereof does not reduce or inhibit one or more of the chaperone proteins Hsp90, Hsp40, Hsp27, HOP or Hsp70. For example, the capsaicin can reduce or inhibit the activity of the Hsp70 chaperone complex as a whole without directly inhibiting Hsp70 itself. Capsaicin caused the specific cellular degradation of Hsp70 and lead to cytotoxicity in cancer cells. In some embodiments capsaicin, or a derivative, analog or prodrug salt thereof can reduce or inhibit Hsp70-mediated folding, activation, assembly, or function of denatured proteins; increase the degradation of Hsp70 complexes including co-chaperones or client proteins or a combination thereof. In some embodiments, capsaicin, or a derivative, analog or prodrug thereof increases apoptosis, reduces proliferation, or a combination thereof.

Therefore, in some embodiments capsaicin, or a derivative, analog or prodrug salt thereof can reduce or inhibit Hsp70-mediated folding, activation, assembly, or function of denatured proteins; increase the degradation of Hsp70 complexes including co-chaperones or client proteins; reduce or inhibit direct association of Hsp70 with death proteins; or a combination thereof. In some embodiments, capsaicin, or a derivative, analog or prodrug thereof increases apoptosis, reduces proliferation, or a combination thereof.

In preferred embodiments, capsaicin, or a derivative, analog or prodrug thereof does not induce or increase expression of Hsp70, Hsp27, Hsp40, or HOP or a combination thereof. In some embodiments, capsaicin, or a derivative, analog or prodrug thereof reduces or decreases the cellular level of Hsp70, Hsp27, Hsp40, or HOP or a combination thereof. In some embodiments, capsaicin, or a derivative, analog or prodrug thereof prevents increase or induction of cellular heat shock responses.

In some embodiments the capsaicin, or a derivative, analog or prodrug thereof binds directly to Hsp70. In some embodiments the capsaicin, or a derivative, analog or prodrug thereof competitively inhibits the binding of other moieties to Hsp70. In other embodiments, capsaicin, or a derivative, analog or prodrug thereof blocks Hsp70 ATPase stimulation by Hsp40.

Generally dosage levels of 0.001 to 50 mg/kg of body weight daily are administered to mammals. Preferably, the dose is 1 to 50 mg/kg, more preferably 1 to 40 mg/kg, or even 1 to 30 mg/kg, with a dose of 2 to 20 mg/kg being also a preferred dose. Examples of other dosages include 2 to 15 mg/kg, or 2 to 10 mg/kg or even 3 to 5 mg/kg, with a dose of about 4 mg/kg being a specific example.

Dosages are commonly in the range of 0.1 to 100 mg/kg, with shorter ranges of 1 to 50 mg/kg preferred and ranges of 10 to 20 mg/kg being more preferred. An appropriate dose for a human subject is between 5 and 15 mg/kg per dose.

In general, by way of example only, dosage forms based on body weight include doses in the range of 5-300 mg/kg, or 5-290 mg/kg, or 5-280 mg/kg, or 5-270 mg/kg, or 5-260 mg/kg, or 5-250 mg/kg, or 5-240 mg/kg, or 5-230 mg/kg, or 5-220 mg/kg, or 5-210 mg/kg, or 20 to 180 mg/kg, or 30 to 170 mg/kg, or 40 to 160 mg/kg, or 50 to 150 mg/kg, or 60 to 140 mg/kg, or 70 to 130 mg/kg, or 80 to 120 mg/kg, or 90 to 110 mg/kg, or 95 to 105 mg/kg, with doses of 3 mg/kg, 5 mg/kg, 7 mg/kg, 10 mg/kg, 15 mg/kg, 20 mg/kg, 25 mg/kg, 30 mg/kg, 50 mg/kg and 100 mg/kg being specific examples of preferred doses. Such doses may, of course, be repeated. The dose will, of course, be correlated with the identity of the mammal receiving said dose. Doses in the above-recited mg/kg ranges are convenient for mammals, including rodents, such as mice and rats, and primates, especially humans, with doses of about 5 mg/kg, about 10 mg/kg and about 15 mg/kg being especially preferred for treating humans.

The compositions can be formulated into unit dose formulation including, but not limited to tablets, capsules, pills, troches or lozenges, cachets, and pellets. The unit dose forms can be provided in packs containing multiple dosages of the compositions. An exemplary pack containing multiple unit dosages includes, but is not limited to blister packs.

3. Controls

The effect of a capsaicin in combination with one or more inhibitors of the Hsp90 pathway can be compared to control. For example, in some embodiments, one or more of the pharmacological or physiological markers or pathways affected by capsaicin treatment is compared to the same pharmacological or physiological marker or pathway in untreated control cells or untreated control subjects. In preferred embodiments the cells or the subject suffers the same disease or conditions as the treated cells or subject. For example, cells or subjects treated with a capsaicin and Hsp90 inhibitor can be compared to cells or subjects treated with other Hsp inhibitors. The cells or subjects treated with other Hsp inhibitors can have a greater increase in Hsp70 expression, Hsp27 expression, or a greater increase in pro-survival signaling than do cells or subjects treated with capsaicin, or a derivative, analog or prodrug thereof.

In some embodiments, the control is cells treated with capsaicin or an inhibitor of the Hsp90 pathway but not the combination thereof.

In preferred embodiments, capsaicin, or a derivative, analog or prodrug thereof in combination with one or more inhibitors of the Hsp90 pathway are effective to reduce, inhibit, or delay one or more symptoms of a cancer in a subject. Cancers that can be treated using the disclosed compositions are discussed in more detail above.

Capsaicin, or a derivative, analog or prodrug, or a pharmacologically active salt thereof and one or more inhibitors of the Hsp90 pathway can be administered enterally or parenterally. Capsaicin, or a derivative, analog or prodrug, or a pharmacologically active salt thereof and one or more inhibitors of the Hsp90 pathway can be part of a pharmaceutical composition that includes a pharmaceutically acceptable carrier.

B. Methods of Treating Other Diseases Using Combination

Therapy

In addition to cancer, the compositions and methods disclosed herein can be used to treat a variety of diseases and disorders in which blockade of the Hsp70 and Hsp90 chaperone pathways is desirable. Hsp70 and Hsp90 are molecular chaperones with important roles in maintaining the functional stability and viability of cells under a transforming pressure. Accordingly, if the Hsp70 and Hsp90 are stabilizing diseased or pathogenic cells, it can be desirable to inhibit the Hsp70 and Hsp90 chaperone pathways and thereby reduce the viability of the diseased or pathogenic cells.

Therefore, the compositions and methods disclosed herein can be used to treat any disease or disorder in which the Hsp70 and Hsp90 chaperone pathway stabilizes or refolds proteins that play a pathogenic role in the diseases or disorder. In some embodiments, Hsp70, Hsp90 or complexes thereof are increased in the cells that express the proteins. Exemplary diseases are provided below.

1. Infectious Diseases

The compositions and methods can be used to treat infectious diseases. Methods of treating an infectious disease including administering to a subject with an infectious disease an effective amount of a naturally occurring capsaicin, synthetic capsaicin, or derivative, analog or prodrug, or pharmacologically active salt thereof to reduce or inhibit one or more symptoms of the infectious disease are disclosed.

a. Bacterial Infections

Hsp70 has been associated with the survival of bacteria under stress conditions (reviewed in Patury, et al., Curr. Top Med. Chem., 15:1337-1351 (2009); Henderson, et al., Infect. Immunol., 74:3693-3706)). Thus, Hsp70 and its co-chaperones are potential drug targets to sensitize prokaryotes to stress, such as antibiotics or host responses.

The prokaryotic Hsp70 analog, DnaK, has been strongly linked to bacterial survival under stress. Consistent with these roles, knockouts of E. coli DnaK are viable under normal laboratory conditions, but exhibit increased sensitivity to elevated temperature or addition of antibiotics. In addition, knockout mutations of DnaK make Staphalococcus aureus less efficient in mouse infection models and Streptococcus mutans DnaKΔ strains have impaired biofilm formation and viability.

The broad-spectrum antibacterial activity of Hsp70 inhibitors and their low propensity for eliciting drug resistance make Hsp70 inhibitors attractive candidates for antibacterial therapy. Therefore, in some embodiments, the compositions and methods disclosed herein are used to treat a disease or disorder associated with a bacteria or bacterial infection. Exemplary bacteria include, but are not limited to, Bacillus spp., Bordetella spp., Borrelia spp., Brucella spp., Campylobacter spp., Chlamydia spp., Clostridium spp., Corynebacterium spp., Escherichia spp., Francisella spp., Haemophilus spp., Helicobacter spp., Legionella spp., Leptospira spp., Listeria spp., Mycobacterium spp., Mycoplasma spp., Neisseria spp., Neisseria spp., Pseudomonas spp., Rickettsia spp., Salmonella spp., Shigella spp., Staphylococcus spp., Staphylococcus spp., Streptococcus spp., Treponema spp., Vibrio spp. and Yersinia spp.

b. Protozoan Infections

Hsp70s have been linked to protozoan infection (Acharya, et al., Mol. Biochem. Parastitol, 153:85-94 (2007)). The analogous molecular chaperone in the human malarial parasite Plasmodium falciparum, PfHsp70, is considered a potential drug target against the parasite (Cockburn, et al., Biol. Chem. 39:431-438 (2011): Chiang et al, Bioorg. Med. Chem., 17:1527-1533 (2009)).

Therefore, in some embodiments, the compositions and methods disclosed herein are used to treat a disease or disorder associated with a protozoan. Exemplary protozoa include, but are not limited to, Trypanosoma spp., Leishmania spp., Giardia spp., Acanthamoeba spp., Balamuthia spp., Entamoeba spp., Babesia spp., Cryptosporidium spp., Cyclospora spp., Plasmodium spp., and Toxoplasma spp.

The compositions can be used to create or rescue drug sensitivity. Therefore, in some embodiments, capsaicin, or a derivative, analog or prodrug, or a pharmacologically active salt thereof is used in combination with a second active agent that is an anti-protozoan.

C. Combination Therapies with Additional Therapeutics

The compositions of capsaicins in combination with one or more inhibitors of Hsp90 disclosed herein can be used in combination with one or more additional therapeutic agents. The term “combination” or “combined” is used to refer to either concomitant, simultaneous, or sequential administration of two or more agents. Therefore, the combinations can be administered either concomitantly (e.g., as an admixture), separately but simultaneously (e.g., via separate intravenous lines into the same subject), or sequentially (e.g., one of the compounds or agents is given first followed by the second). The additional therapeutic agents can be administered locally or systemically to the subject, or coated or incorporated onto, or into a device.

The additional agent or agents can modulate the Hsp70 chaperone pathway, the Hsp90 pathway, or the capsaicin or Hsp90 inhibitor itself. For example, the additional agent can enhance or reduce the activity of the Hsp70 or Hsp90 chaperone pathways, or the capsaicin or Hsp90 inhibitor itself. The additional agent or agents can be a second therapeutic that is used to enhance the therapeutic effect of capsaicin, or a derivative, analog or prodrug, or a pharmacologically active salt thereof in combination with an Hsp90 inhibitor by targeting a second molecular pathway relevant to the disease, disorder, or condition being treated. In some embodiments, the one or more additional agent is a conventional therapeutic agent for the disease, disorder, or condition to be treated. For example, if the disease to be treated is cancer, a conventional therapeutic agent can be chemotherapy.

It is believed that Hsp70 and Hsp90 inhibitors can be used to increase the sensitivity of target cells to some conventional therapeutic agents. Therefore, in some embodiments, the second (conventional) therapeutic agent is used at a lower dosage or for a shorter duration than if it used alone. For example, if capsaicin, or a derivative, analog or prodrug, or a pharmacologically active salt thereof in combination with one or more Hsp90 inhibitors is administered in combination with a chemotherapeutic agent to target cancer cells, the chemotherapeutic agent can be used at lower dosage or for a shorter duration than if the chemotherapeutic agent is administered without capsaicin, or a derivative, analog or prodrug, or a pharmacologically active salt thereof in combination with one or more Hsp90 inhibitors.

1. Additional Chemotherapeutic Agents

Additional therapeutic agents can also include conventional cancer therapeutics such as chemotherapeutic agents, cytokines, chemokines, and radiation therapy. The majority of chemotherapeutic drugs can be divided into: alkylating agents, antimetabolites, anthracyclines, plant alkaloids, topoisomerase inhibitors, and other antitumor agents. All of these drugs affect cell division or DNA synthesis and function in some way. Additional therapeutics include monoclonal antibodies and the tyrosine kinase inhibitors e.g., imatinib mesylate (GLEEVEC® or GLIVEC®), which directly targets a molecular abnormality in certain types of cancer (chronic myelogenous leukemia, gastrointestinal stromal tumors).

In a preferred embodiment the additional therapeutic agent is a chemotherapeutic agent. Representative chemotherapeutic agents include, but are not limited to cisplatin, carboplatin, oxaliplatin, mechlorethamine, cyclophosphamide, chlorambucil, vincristine, vinblastine, vinorelbine, vindesine, taxol and derivatives thereof, irinotecan, topotecan, amsacrine, etoposide, etoposide phosphate, teniposide, epipodophyllotoxins, trastuzumab (HERCEPTIN®), cetuximab, and rituximab (RITUXAN® or MABTHERA®), bevacizumab (AVASTIN®), and combinations thereof.

2. Drugs to Treat Infection

In some embodiments the additional therapeutic agents are agents that treat infection, such as antibacterial and anti-protozoan drugs. Combination therapy with Hsp70 and Hsp90 inhibitors provides means for improving treatment of bacterial disease because it reduces the emergence of drug resistance. In one embodiment the additional therapeutic agent is an antibiotic. Representative antibiotics include, but are not limited to Ampicillin, Bacampicillin, Carbenicillin Indanyl, Mezlocillin, Piperacillin, Ticarcillin, Amoxicillin, Ampicillin, Benzylpenicillin, Cloxacillin, Dicloxacillin, Methicillin, Oxacillin, Penicillin G, Penicillin V, Piperacillin, Ticarcillin, Nafcillin, Cephalosporin Cefadroxil, Cefazolin, Cephalexin, Cephalothin, Cephapirin, Cephradine, Cefaclor, Cefamandol, Cefonicid, Cefotetan, Cefoxitin, Cefprozil, Ceftmetazole, Cefuroxime, Loracarbef, Cefdinir, Ceftibuten, Cefoperazone, Cefixime, Cefotaxime, Cefpodoxime proxetil, Ceftazidime, Ceftizoxime, Ceftriaxone, Cefepime, Azithromycin, Clarithromycin, Clindamycin, Dirithromycin, Erythromycin, Lincomycin, Troleandomycin, Cinoxacin, Ciprofloxacin, Enoxacin, Gatifloxacin, Grepafloxacin, Levofloxacin, Lomefloxacin, Moxifloxacin, Nalidixic acid, Norfloxacin, Ofloxacin, Sparfloxacin, Trovafloxacin, Oxolinic acid, Gemifloxacin, and Pefloxacin.

3. Other Active Agents

Other active agents that can be used alone, or in combination with capsaicin include, but are not limited to, vitamin supplements, appetite-stimulating medications, medications that help food move through the intestine, nutritional supplements, anti-anxiety medication, anti-depression medication, anti-coagulants, clotting factors, antiemetic medications, antidiarrheal medications, anti-inflammatories, steroids such as corticosteroids or drugs that mimic progesterone, omega-3 fatty acids supplements, eicosapentaenoic acid supplements, anti-inflammatories, anabolic agents, psycho-stimulants, selective androgen-receptor modulators, anti-depressant medications, anti-anxiety medications and analgesics.

D. Therapeutic Administration

Pharmaceutical compositions including capsaicin, or a derivative, analog or prodrug, or a pharmacologically active salt thereof in combination with one or more Hsp90 inhibitors, may be administered in a number of ways depending on whether local or systemic treatment is desired, and depending on the area to be treated. For example, the disclosed compositions can be administered intravenously, intraperitoneally, intramuscularly, subcutaneously, intracavity, or transdermally. The compositions may be administered orally, parenterally (e.g., intravenously), by intramuscular injection, by intraperitoneal injection, transdermally, extracorporeally, ophthalmically, vaginally, rectally, intranasally, topically or the like, including topical intranasal administration or administration by inhalant. Parenteral administration of the composition, if used, is generally characterized by injection. Injectable compositions can be prepared in conventional forms, either as liquid solutions or suspensions, solid forms suitable for solution of suspension in liquid prior to injection, or as emulsions. A revised approach for parenteral administration involves use of a slow release or sustained release system such that a constant dosage is maintained.

For all of the disclosed compounds, as further studies are conducted, information will emerge regarding appropriate dosage levels for treatment of various conditions in various patients, and the ordinary skilled worker, considering the therapeutic context, age, and general health of the recipient, will be able to ascertain proper dosing. The selected dosage depends upon the desired therapeutic effect, on the route of administration, and on the duration of the treatment desired. Generally dosage levels of 0.001 to 100 mg/kg of body weight daily are administered to mammals. Generally, for intravenous injection or infusion, dosage may be lower. Preferably, the compositions are formulated to achieve a capsaicin serum level of about 1 to about 1000 μM.

In a preferred embodiment, 50, 20 or 10 mg/kg of 17-AAG are used in combination with 2.5 or 5 mg/kg of capsaicin.

### EXAMPLES

### Example 1

### Capsaicin Inhibits the Hsp90/Hsp70 Chaperoning Pathway

### Materials and Methods

Progesterone Receptor (PR) Reconstitution Assay

To identify small molecule inhibitors of molecular chaperones, a high-throughput functional screen was developed based on the isoform A of progesterone receptor (PRA), a well-established physiological client of Hsp90, and using rabbit reticulocyte lysate (RRL) as a source of molecular chaperones. This comprehensive functional assay measured the recovery of hormone binding activity of PRA after mild heat treatment. Purified PR was adsorbed onto PR22 antibody bound to protein A that was absorbed on 96 well plates. 100 μl of RRL lysate and ATP regeneration system was added to each well. After incubation for 30 min at 30° C., 0.1 μM [3H]-progesterone (American Radiolabeled Chemicals, Inc #ART 0063) was added. Plates were incubated on ice for 2 h at 4° C. Complexes were then washed three times with 200 μl of reaction buffer (20 mM Tris/HCl, pH 7.5, 5 mM MgCl₂, 2 mM DTT, 0.01% NP-40, 50 mM KCl and 5 mM ATP) and assessed for bound progesterone by liquid scintillation using PerkinElmer Microbeta plate reader.

### Results

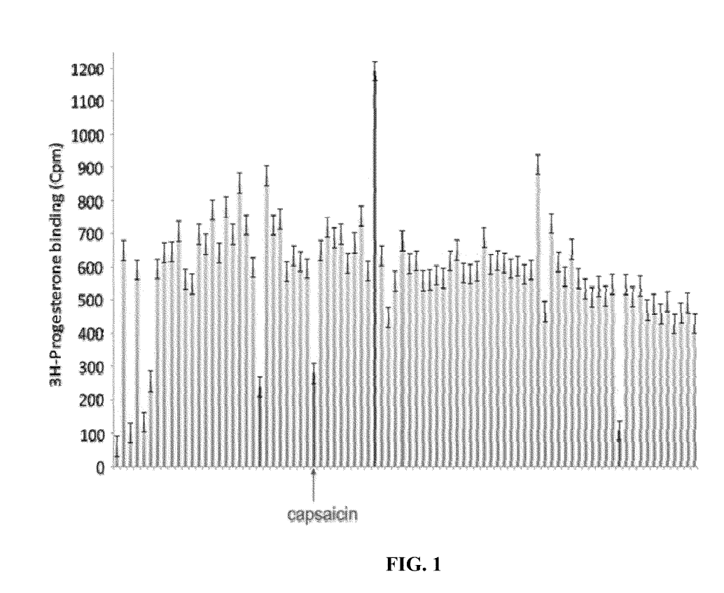

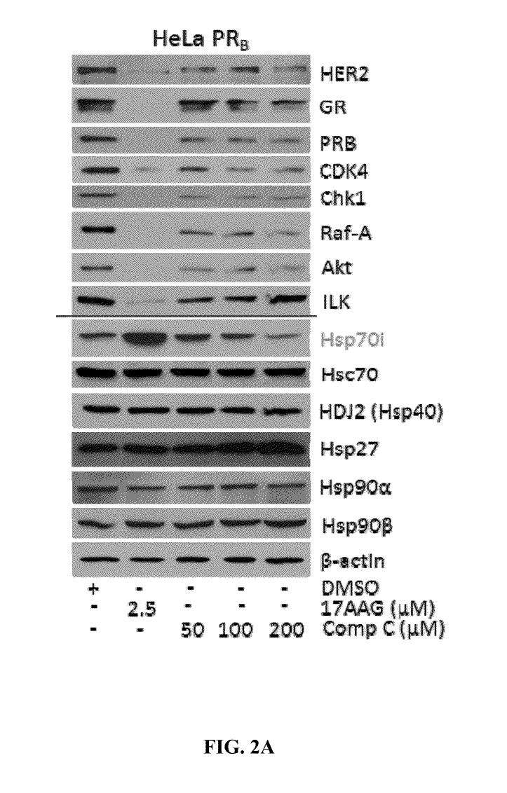

To identify small molecule inhibitors of Hsp90 and its co-chaperones, a high throughput functional screen based on the progesterone receptor (PR), was developed. This assay was used to screen a compound library from the NIH Clinical Collection. One inhibitor that showed efficient and reproducible modulation of the Hsp90 chaperoning activity was identified as capsaicin (FIG. 1).

### Example 2

### Capsaicin Reduced the Level of Hsp70 in all Tested Cancer Cell Lines

### Materials and Methods

Western Blotting

Cells were lysed with buffer A (10 mM Tris pH 8.0, 137 mM NaCl, 10% glycerol, 1% Nonident P-40) supplemented with protease inhibitor cocktail (Roche Applied Science catalog no. 11 836 170 001) on ice for 30 minutes, shaking every 5 minutes. Lysate was centrifuged at 16,000×g for 10 minutes. 10 μg of clarified lysates were run on SDS-PAGE (10% gel) and transferred to PVDF membrane. Proteins were detected by Western blotting using antibodies against Hsp90β (H90.10), Hsp90α (D7α), Hsp70 from Enzo Life sciences (catalog no. ADI-SPA-810), HSC70 from StressMarq Biosciences Inc (catalog no. SMC-151), Hop (F5), Hsp40 from Neomarkers (catalog no. KA2A5.6), Hsp27 from StressMarq Biosciences Inc (catalog no. SMC-161), p23 (B3), GR (home-made GR), PR_{B} (PR6), AR from Santa Cruz Biotechnology, Inc (catalog no. sc-816), HER2 from Cell Signaling (catalog no.#4290), Raf-A from Santa Cruz Biotechnology, Inc (catalog no. 166771), Chk1 from Santa Cruz Biotechnology, Inc (catalog no. sc-8408), CDK4 from Santa Cruz Biotechnology, Inc (catalog no. sc-260), ILK from BD Biosciences (catalog no. 611803), Akt from Cell Signaling (catalog no.#4691P), LC3B from Cell Signaling (catalog no.#3868), β-actin from Santa Cruz Biotechnology, Inc (catalog no. sc-477786).

### Results

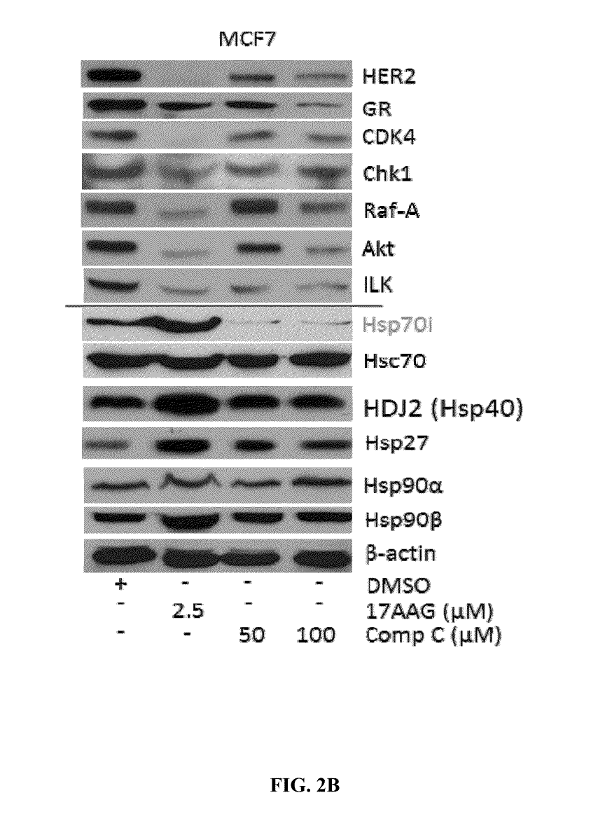

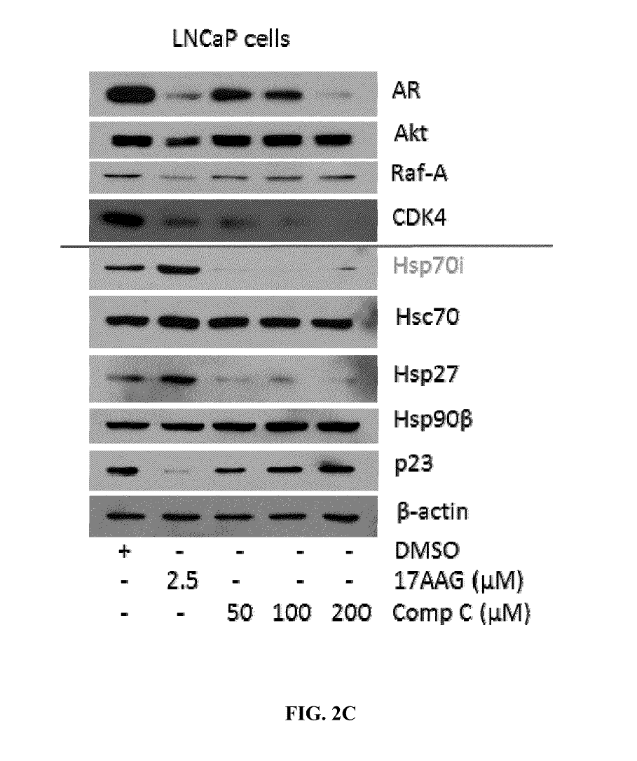

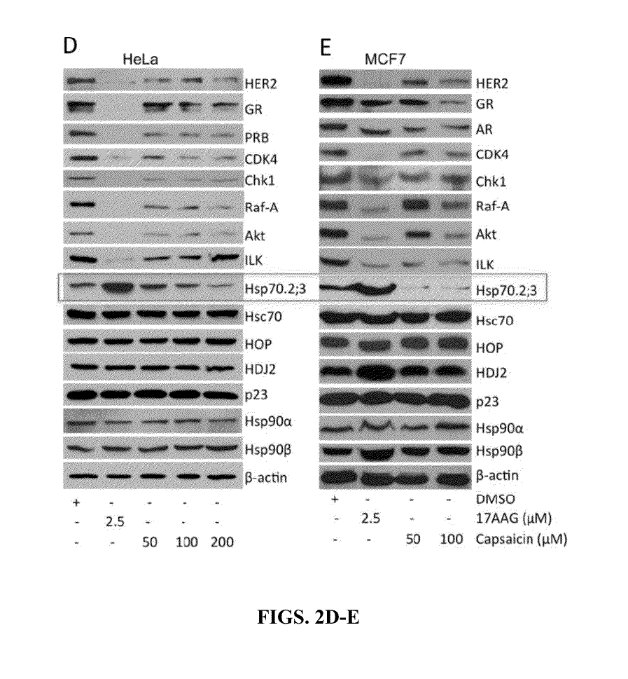

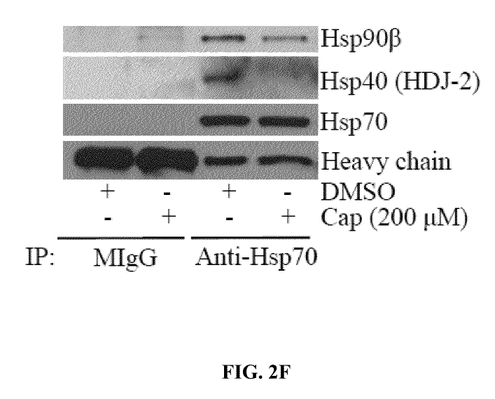

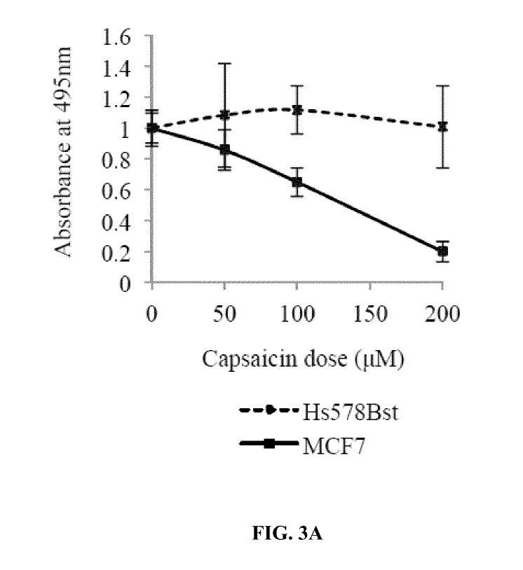

The impact of capsaicin on the Hsp90 molecular signature was evaluated. As shown in FIGS. 2A-F, capsaicin treatment caused cellular degradation of several kinase protein clients of Hsp90 (Her2, CDK4, ChK1, Raf-A, Akt and ILK) and steroid receptors: glucocorticoid receptor (GR), PR and AR. Capsaicin did not affect the expression level of molecular chaperones such as Hsp90α, Hsp90β, Hsp40, p23 and Hop. Compared to 17-AAG, the canonical inhibitor of Hsp90, capsaicin did not induce overexpression of Hsp70. On the contrary, capsaicin reduced the level of Hsp70 in HeLa and MCF7 cells. This effect was specific to the inducible form of Hsp70 (Hsp70i). The level of the closely related Hsc70, the constitutive form, did not change upon capsaicin treatment in either cell line.

### Example 3

### Capsaicin Selectively Kills Cancer Cells and Induces Autophagy in MCF7 and LNCaP Cells

### Materials and Methods

Colony Formation Assay

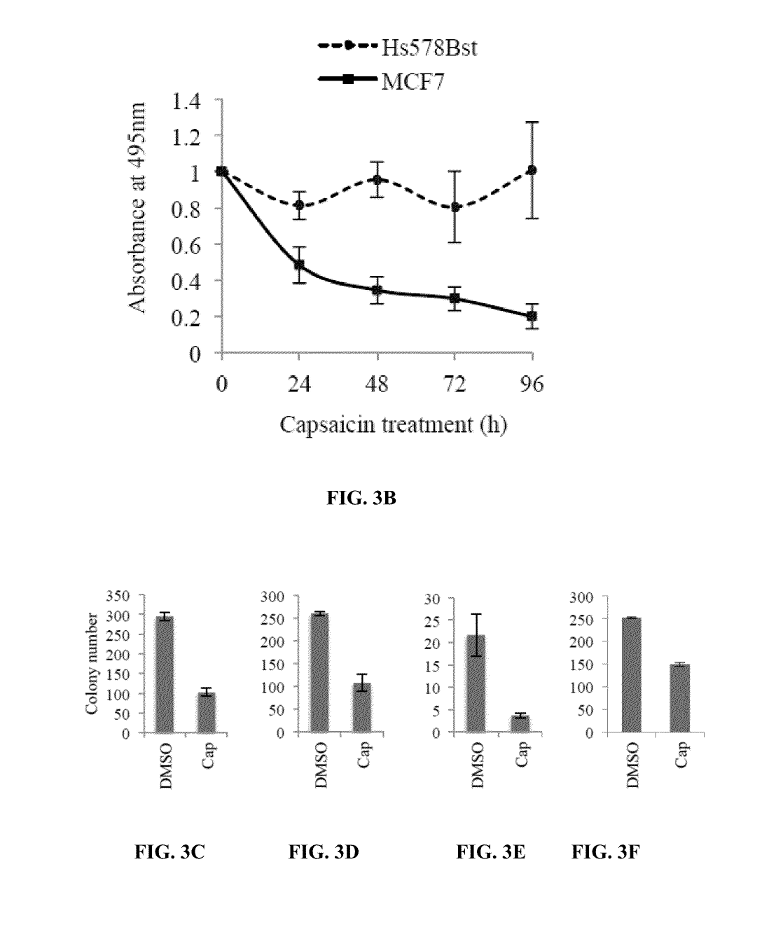

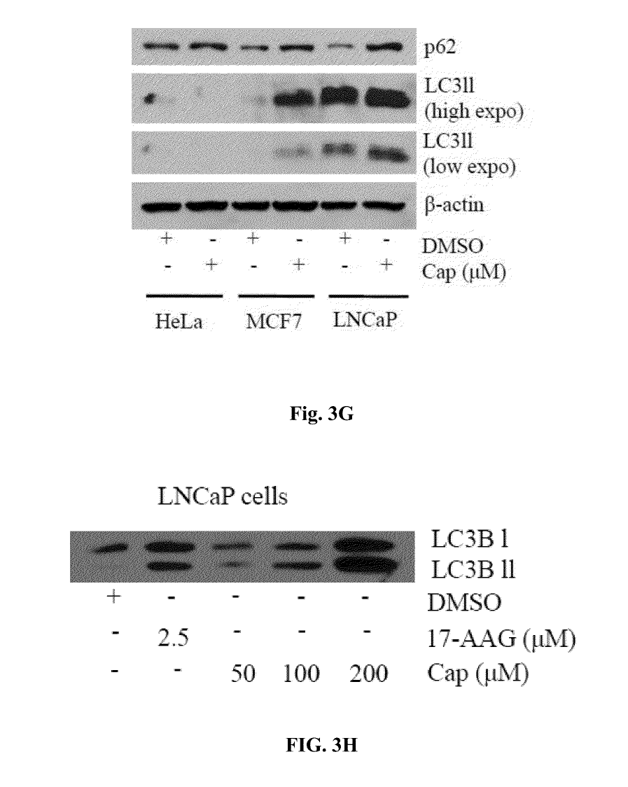

Cells were grown in 6-well tissue culture plates to 60% confluence and treated with 200 μM capsaicin or DMSO control for 24 h. Cells were then collected and 1000 of these cells were re-plated per 10 cm tissue culture dish (Falcon, catalog no. 353003) in triplicate experiments. Cells were grown for 15 days in MEM 1× media supplemented with 10% FBS. Cells were fixed with 6% Glutaraldehyde and 0.5% Crystal Violet, and colonies that contained 50 cells were counted. (see FIGS. 3C-F and 4A-4C)

MTT Cell Proliferation Assay

3,000 cells were plated on 96-well tissue culture plates (Corning, catalog no. 3599) and grown to 60% confluence before treatment with indicated concentrations of 17-AAG, Capsaicin or DMSO control (2% total DMSO concentration) for indicated time. Cells were incubated with 10 μl of The CellTiter 96® AQueous One Solution Cell Proliferation Assay reagent (Promega, catalog no. G3580) and 90 μl of culture media/well for 1 h at 37° C. Absorbance at 495 nm was measured using SAFIRE-TECAN plate reader.

### Results

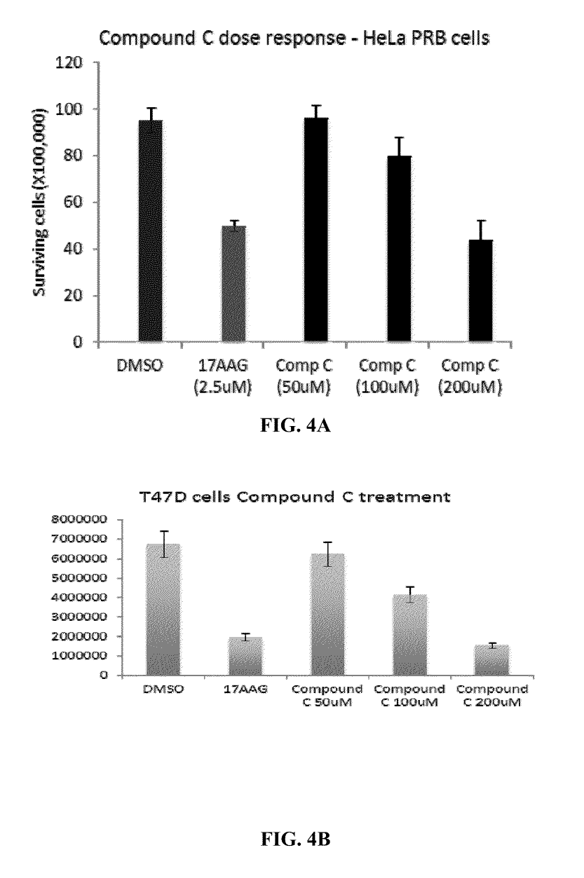

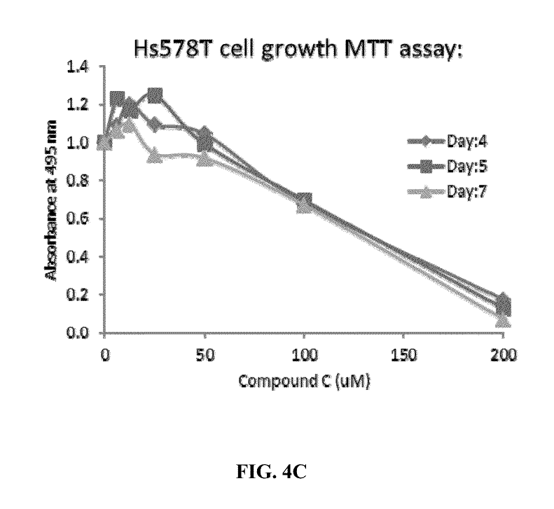

Data in FIGS. 3A and 3B and 4C show capsaicin caused cell death of MCF7 cancer cells, as opposed to normal mammary epithelial cells Hs578Bst, in a concentration and time-dependent manner.

As shown in FIGS. 3C-3F, capsaicin efficiently reduces the ability of 6 cancer cells to form colonies. Further studies to understand the mechanism of cell death induced by capsaicin revealed that capsaicin induces autophagy in MCF7 and LNCaP cell lines. When autophagy is triggered, LC3 (microtubuleassociated protein 1 light chain 3) is cleaved to generate LC3I, which is further lipidated to form LC3II. LC3II is then integrated into budding autophagosomal membranes. Increased LC3II protein level is a reliable marker of ongoing macroautophagy The data using MCF7 and LNCaP cells showed that LC3II levels were significantly increased in both cell lines after treatment with capsaicin (FIG. 3G). However, HeLa cells showed no LC3II modification. FIG. 3H shows concentration-dependent induction of LC3B modification in LNCaP cells after 24 h treatment. These cells may undergo apoptosis as indicated by cleavage, albeit low, of PARP (data not shown). Light microscopic analysis of MCF7 cells showed that capsaicin-treated cells harbor a large accumulation of vacuoles (date not shown). Ultra-structural examination of these cells using transmission electron microscopy revealed that these vacuoles have a double membrane lining indicative of autophagy (data not shown). Mitochondria in capsaicin-treated cells were swollen, with irregular membranes. The normal pattern of mitochondrial cristae (inner mitochondrial membrane) was lost. Some of these vacuoles also formed cup-shaped structures that engulfed unhealthy mitochondria, thus revealing an active mito-autophagic process (data not shown). These findings strongly correlate with two recent reports showing that capsaicin treatment induces macro-autophagy in MCF7 breast cancer cells and that dihydrocapsaicin (a saturated analogue of capsaicin) induces mitoautophagy in HCT 116 colon cancer cells. To date, no direct correlation is found between capsaicin-induced autophagy and its inhibition of Hsp90/Hsp70 chaperone machinery.

The concentrations used are comparable to what is reported in the literature. The fact that capsaicin inhibited chaperoning of PR in vitro using the 5P-system indicates that capsaicin interferes with the activity of the Hsp90 chaperoning machine. This is in line with findings showing that capsaicin destabilizes AR, survivin and HER-2, and interferes with β-catenin, PI3K/Akt, FAK/Akt and Raf/ERK pathways (see FIGS. 2A-E). Importantly, all these signaling proteins are well-documented clients of the Hsp90/Hsp70 chaperoning machine. Thus, the seemingly diverse effects of capsaicin converge to the inhibition of the Hsp90/Hsp70 chaperoning pathway.

### Example 4

### Capsaicin Interacts Directly with the Hsp70 Protein

### Materials and Methods

Gene Expression Assay

50,000 MCF7 cells were plated on 6-well tissue culture plates (Corning, catalog no. 3516) and grown to 60% confluence. Cells were treated with indicated concentrations of 17-AAG, Capsaicin and DMSO control (2% total DMSO concentration) for 24 h. Cells were then harvested, RNA isolated using (Qiagen, catalog no.74104) and reverse transcriptase PCR was done using two-step RT-PCR kit (Qiagen, catalog no. 205920). Hsp70 primers used:

Hsp70F: GGCTGCAGGCACCGGCGCGTCG
HSP70R: CGGTGTTCTGCGGGTTCAGCGC

Fluorescence Emission Assay

To determine the effect of molecular chaperones on the fluorescence emission of capsaicin, all chaperones were tested at 15 μM and capsaicin at 7.5 μM concentrations. Proteins were incubated for 1 h at room temperature in the presence or absence of capsaicin. A 96-well black plate and a SAFIRE-TECAN plate reader were used. The excitation wavelength was 266 nm.

### Results

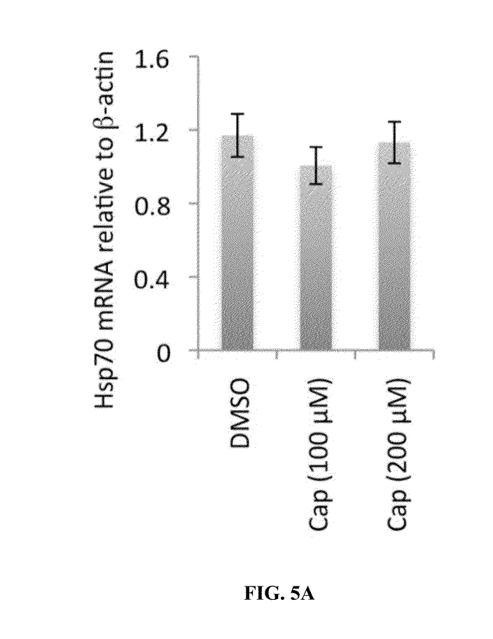

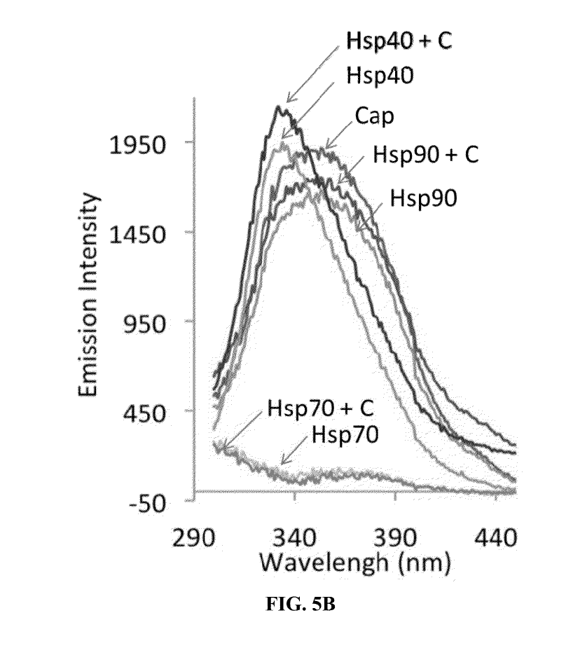

Capsaicin-mediated down-regulation of Hsp70 (see FIGS. 2A-E) could be the consequence of the repression of Hsp70 gene transcription or destabilization of Hsp70 at the protein level. RT-PCR was used to assess the level of Hsp70 mRNA after capsaicin treatment. No change in the level of Hsp70 mRNA was observed, indicating that capsaicin causes a decrease of Hsp70 protein (FIG. 5A). Because capsaicin may resemble a hydrophobic peptide, it was reasoned that capsaicin may bind to Hsp70, causing its cellular degradation. Whether Hsp70 directly binds to capsaicin in vitro using purified Hsp70 was tested by monitoring the fluorescence emission spectrum of capsaicin after excitation at 266 nm. As shown in FIG. 5B, the presence of two-fold concentration of Hsp70 completely quenched capsaicin fluorescence emission, indicating a direct interaction between Hsp70 and capsaicin in solution. An attempt to use other chaperones, Hsp90 and Hsp40, as controls seemed to indicate that they may not significantly change the capsaicin fluorescence emission. However, these two chaperones have maximum emission at 340-350 nm, which may interfere with this interpretation.

### Example 5

### Evaluation of a Combinatorial Therapy with 17-AAG and Capsaicin In Vitro and In Vivo

### Results

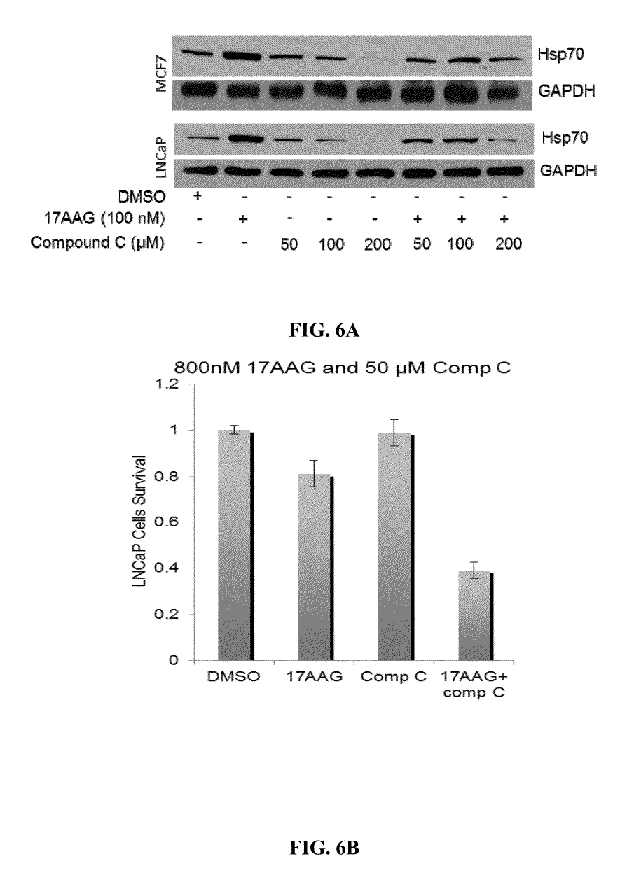

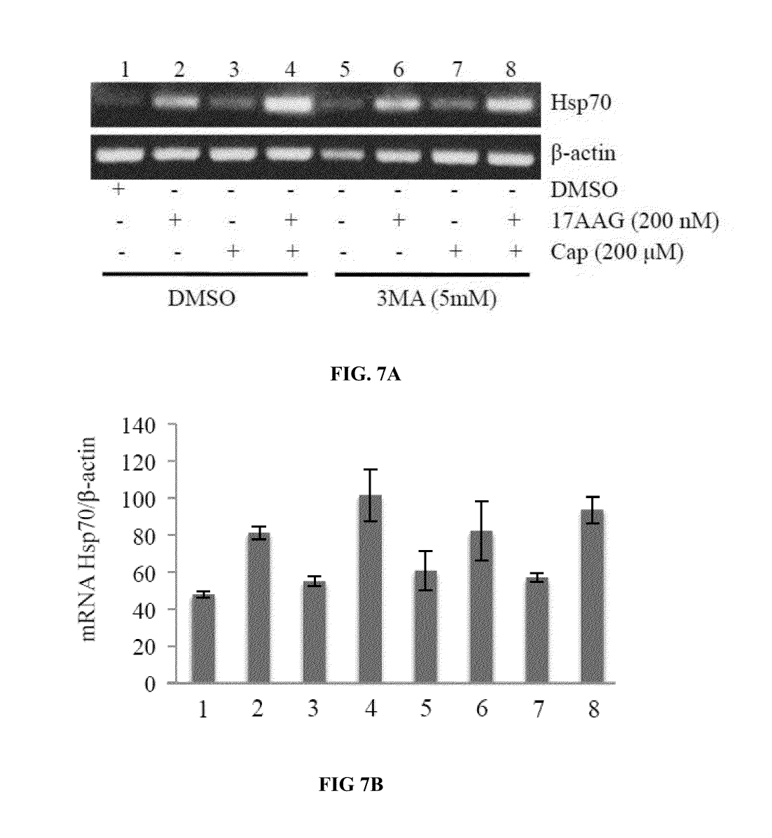

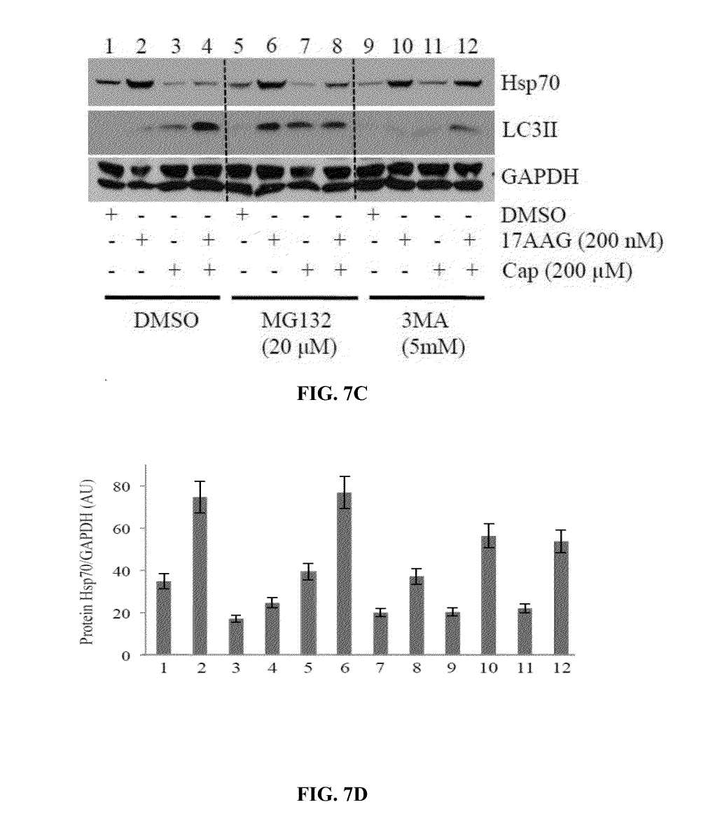

As shown in FIGS. 6A-6B and 7A-7C, combination of capsaicin and 17-AAG abrogated the 17-AAG-induced overexpression of Hsp70 in LNCaP and MCF7 cells, indicating an increased effect of these two drugs in killing cancer cells. This concept is supported by the data in FIG. 6B showing that although individual treatment of LNCaP cells with 17-AAG or capsaicin have very little effect or no effect, combination of both drugs efficiently kills the cells.

### Example 6

### Capsaicin Induces Lysosomal (or Autophagic) Degradation of Hsp70

### Results

Capsaicin induces lysosomal (or autophagic) degradation of Hsp70: Capsaicin-mediated downregulation of Hsp70, (data not shown), could be the consequence of repression of Hsp70 gene transcription, translation or destabilization of Hsp70 at the protein level. RT-PCR was used to assess the level of Hsp70 mRNA after capsaicin treatment. The level of Hsp70 mRNA was slightly increased after capsaicin treatment (FIG. 7A, lanes 1 and 3), yet the protein level was decreased (FIG. 7C, lanes 1 and 3). Importantly, the combination of capsaicin and 17-AAG induced a remarkable increase of Hsp70 mRNA expression (FIG. 7A, lane 4). The protein level in contrast has decreased dramatically (FIG. 7C, compare lanes 2 and 4). These data confirmed that capsaicin abrogates Hsp70 over-expression induced by 17-AAG and also suggest that capsaicin causes destabilization of Hsp70 at protein level. To test whether capsaicin-induced degradation of Hsp70 protein is carried out by proteasome or lysosome/autophagy pathways, cells were treated with capsaicin in presence of the proteasome inhibitor (MG132) or the autophagy inhibitor (3MA) (FIG. 7). As shown in FIG. 7C (lanes 5 and 7), 6 h treatment with MG132 does not rescue Hsp70 when capsaicin alone is used. It also provides a minimal stabilization when capsaicin is used in combination with 17-AAG (FIG. 7C lanes 6 and 8). However, treatment with 3MA for 6 h almost fully stabilizes Hsp70 (FIG. 7C lanes 10 and 12), indicating that Hsp70's degradation is mainly mediated by autophagy. This correlates with the fact that capsaicin induces cell death with autophagy in LNCaP and MCF7 (FIGS. 3G and 3H). The overall profile of Hsp70 mRNA remains very similar with and without 3MA treatment (FIGS. 7B and 7D).

In the same set of samples, a stronger LC3II signal in capsaicin and 17-AAG combinatorial treatment was observed as compared to capsaicin alone (FIG. 7C), suggesting that capsaicin mediated autophagy was further accentuated by 17-AAG. This increased autophagy might help explain the drastic loss of Hsp70 protein levels in the capsaicin-17-AAG co-treatment group, even though the Hsp70 mRNA level is higher compared to control. As expected, 3MA reduced LCII accumulation (FIG. 7C). Taken together, these data (FIG. 7A, 7C) strongly suggest that co-treatment of capsaicin with 17-AAG stimulates the autophagic machinery, which further promotes efficient degradation of Hsp70 protein via the lysosome/autophagy pathway. Molecular mechanisms underlying this process are however still unclear. Further investigations are needed to understand how the autophagy originally started by capsaicin treatment is further intensified by 17-AAG treatment.

To further characterize the autophagy induced by capsaicin we examined the fate of another protein marker of autophagy; the adaptor/scaffold protein p62/SQSTM1. The balanced protein level of p62/SQSTM1 is tightly regulated by autophagy. Its degradation is considered a marker of active autophagy and its accumulation indicates inhibition of defective autophagic degradation. Intriguingly, western blot analysis showed that capsaicin treatment caused accumulation of p62/SQSTM1 (FIGS. 4G, 4H). This would indicate that even though capsaicin triggers autophagy, it is not fully executed.

### Example 7

### High-Throughput Screening of NIH Clinical Collection Drug Library

Methods

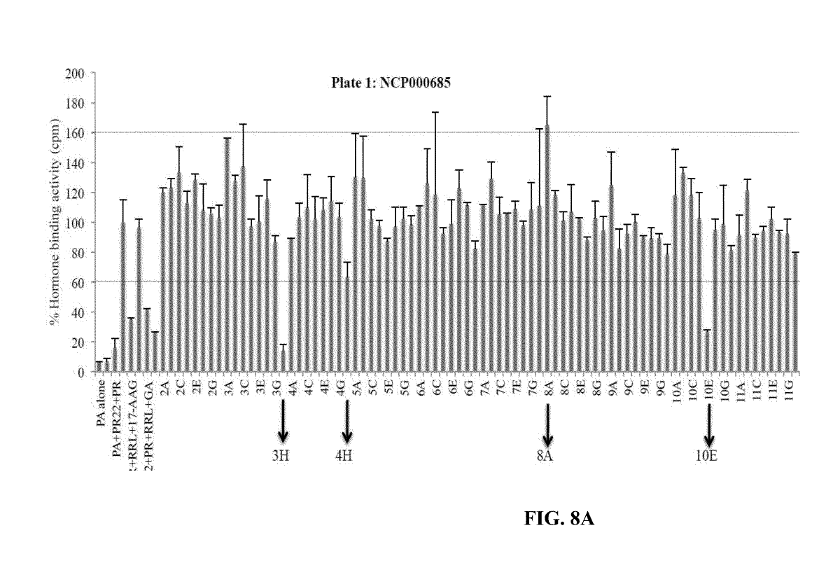

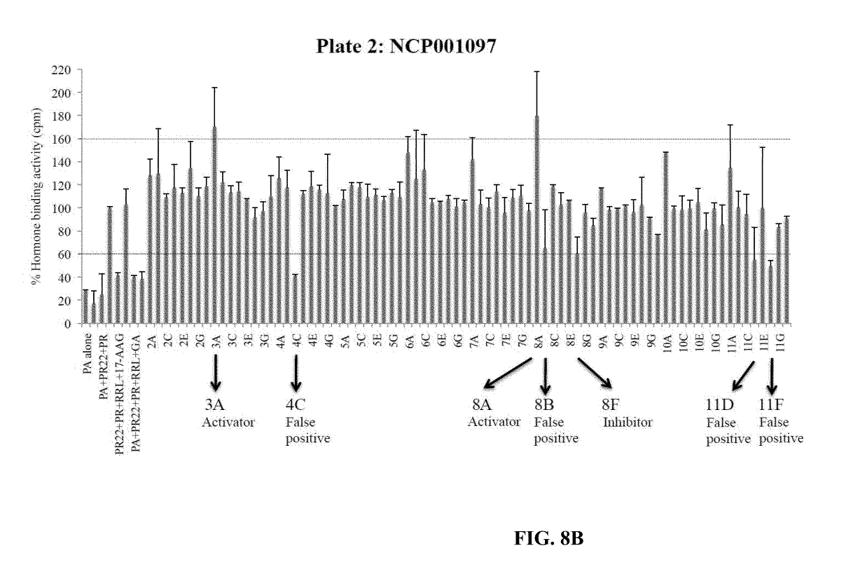

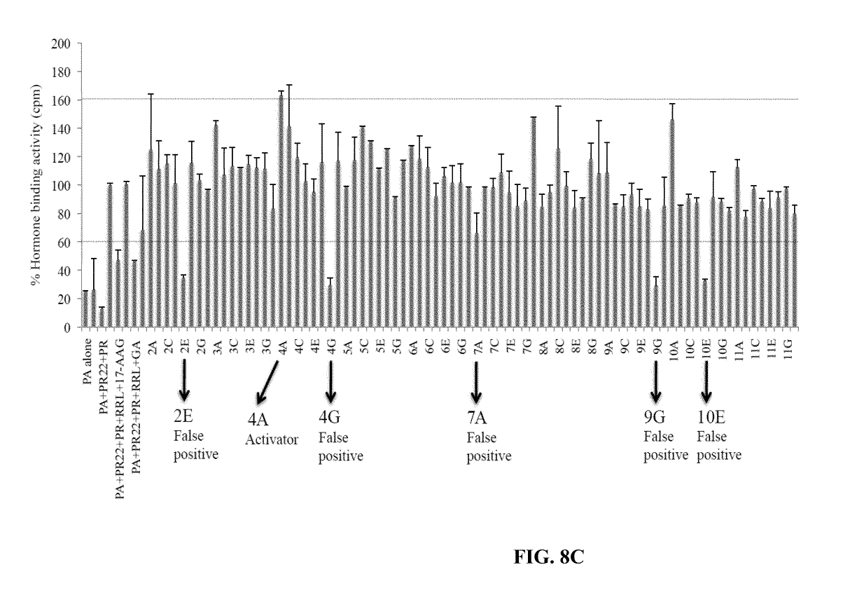

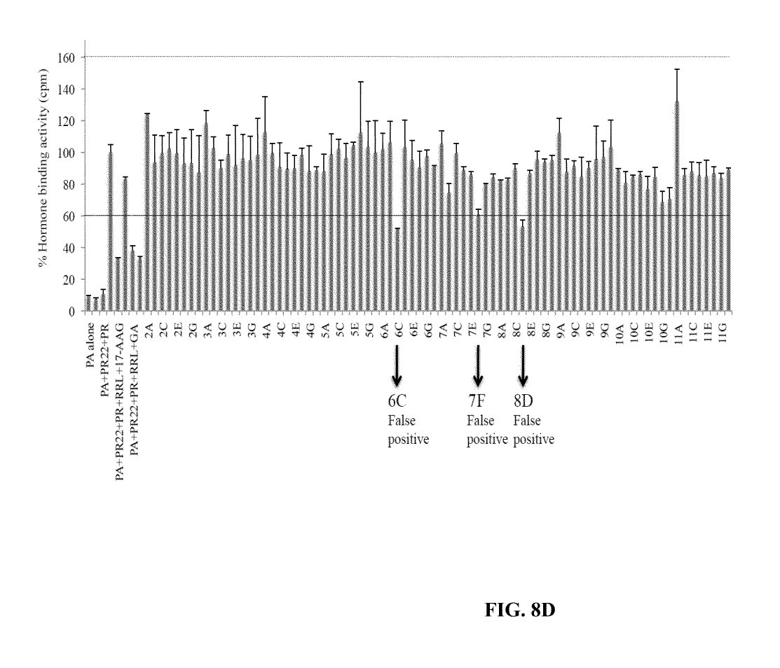

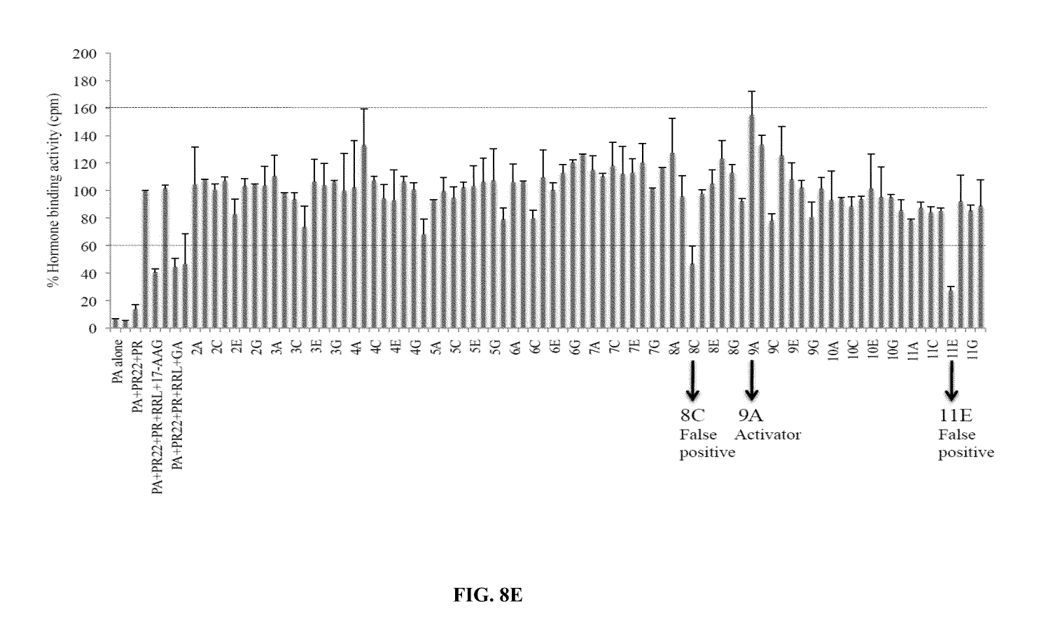

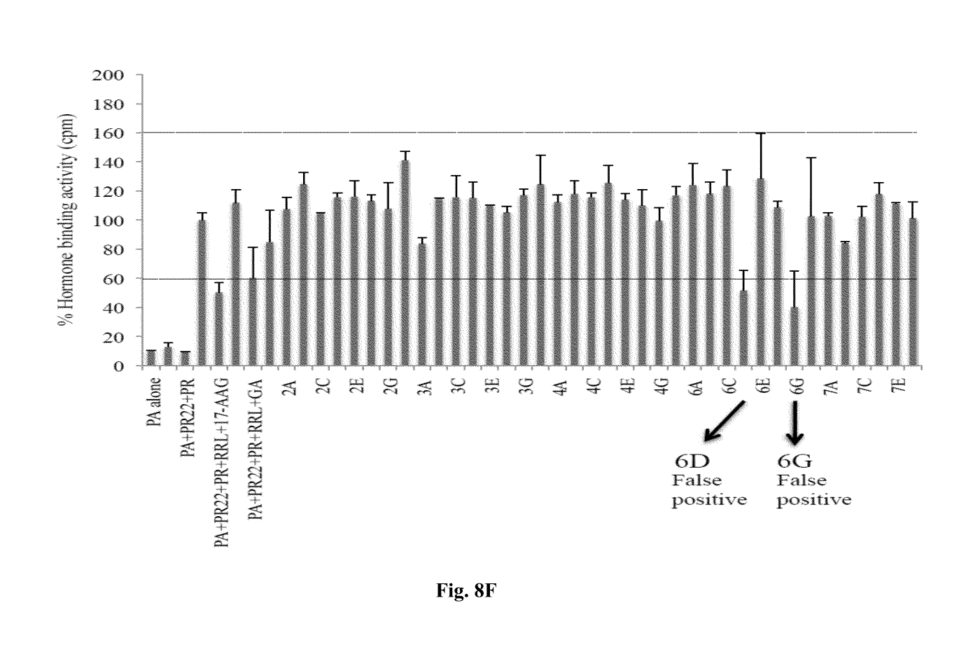

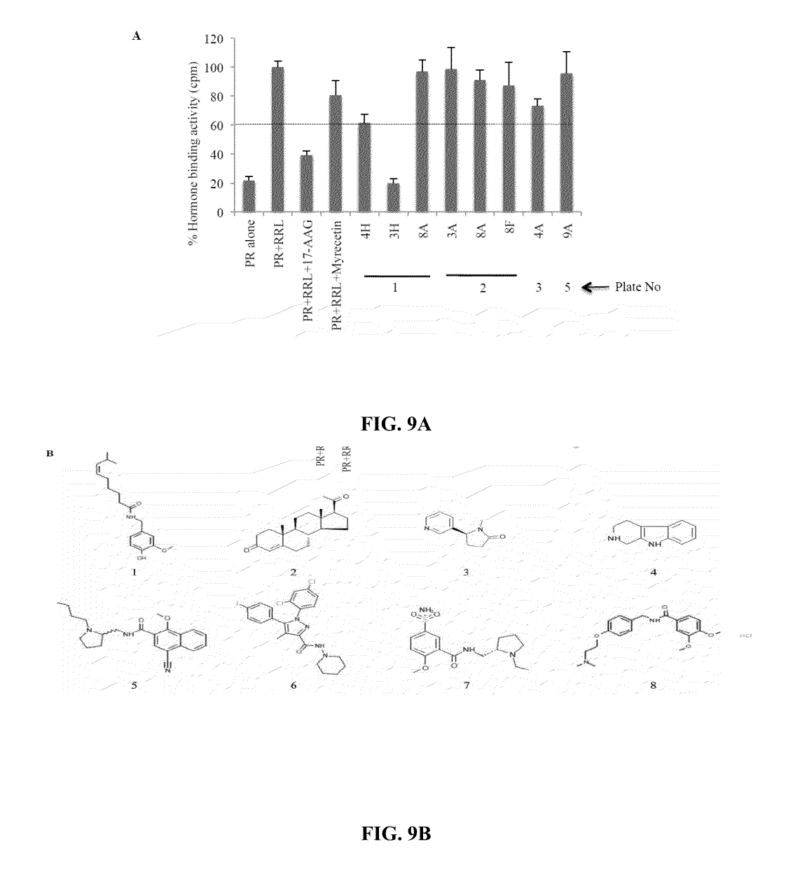

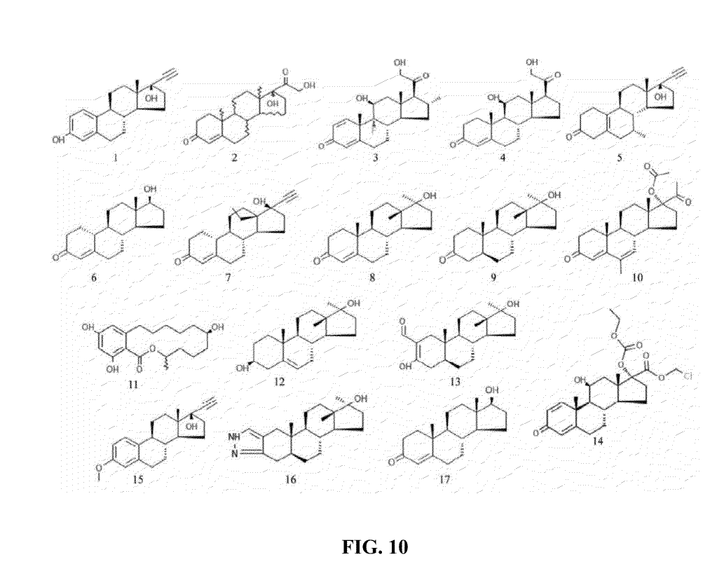

PR complexes were reconstituted on a 96-well plate; the first eight samples contain the following internal controls: 1. Protein A alone, 2. Protein A+PR22, 3. Protein A+PR22+PR, 4. Protein A+PR22+PR+RRL, 5. Protein A+PR22+PR+RRL+17-AAG (20 μM), 6. Protein A+PR22+PR+RRL+Myrecetin (20 μM), 7. Protein A+PR22+PR+RRL+geldanamycin (20 μM), 8. Protein A+PR22+PR+RRL+gedunin (20 μM). PR22 is a monoclonal antibody against Avian PR. The remaining compounds were obtained from the following plates: NCP000685 (FIG. 8A), NCP001097, (FIG. 8B) NCP000800, (FIG. 8C) NCP000899, (FIG. 8D) NCP000998, (FIG. 8E) NCP001169 (FIG. 8F). Primary hits are represented with arrows on the x-axis. Inhibitors and activators that are non-steroidal compounds are listed in Table 1. The chemical structures for the compounds of Table 1 are provided in FIG. 9B. False positive hits (steroidal compounds) are listed in Table 2. The chemical structures for the compounds of Table 2 are provided in FIG. 10. The standard deviation of duplicate samples is shown as error bars.

In FIG. 9A compound hits from indicated plates were re-screened at 20 μM final concentration. The standard deviation of triplicate samples is shown as error bars. Plate no. 1. NCP000685, 2. NCP001097, 3. NCP000800, 5. NCP000998. The first four wells are internal controls: 1. Protein A+PR22+PR, 2. Protein A+PR22+PR+RRL, 3. Protein A+PR22+PR+RRL+17-AAG (20 μM), 4. Protein A+PR22+PR+RRL+Myrecetin (20 μM). Progesterone was used as a positive control.

Results

Primary hits are represented with arrows on the x-axis (FIGS. 8A-8E). Inhibitors and activators that are non-steroidal compounds are listed in Table 1 and depicted in FIG. 9B. False positive hits (steroidal compounds) are listed in Table 2. The structures of the compounds of Table 2 are provided in FIG. 9C. The standard deviation of duplicate samples is shown as error bars.

PR is a physiological client of the Hsp90/Hsp70 chaperone system in cells. Seminal work from the laboratories of David Toft, William Pratt, David Smith, and other researchers has led to the reconstitution of PR chaperone complexes in vitro using either rabbit reticulocyte lysate (RRL), which serves as a complete source of molecular chaperones, or five purified chaperones (Hsp90, Hsp70, HOP, Hsp40 (Ydj), and p23) as a minimal system essential for chaperoning steroid receptors. Properly folded PR binds progesterone with high affinity. The assay is a protein A-sepharose resinbased immuno-precipitation assay where recombinant avian PR multi-chaperone complexes are isolated from SF9 cells using the specific monoclonal antibody PR22. PR is then stripped from SF9 endogenous associated proteins with a high salt buffer. Incubation of the naked PR at 30° C. leads to total loss of PR hormone-binding activity in less than five min. However, adding RRL in the presence of an ATP regeneration system reconstitutes PR multi-chaperone complexes and preserves the hormone binding activity for as long as 30 min at 30° C. (not shown). This assay therefore reflects the ability of Hsp90 and its co-chaperones to protect/refold partially heat-denatured PR to its ligand-binding conformation.

This assay was transformed to a 96-well plate format, thus increasing the throughput of the assay. In this format, the assay has 0.723 Z′ factor value, more than five times signal-to-noise ratio, and 10.7% overall standard deviation. The assay is thus robust enough and can be used as a reliable tool for high-throughput drug screening.

A small chemical library consisting of 446 compounds called ‘NIH clinical collection’ was screened. This library consists of FDA-approved drugs or compounds that are in Phase I, II, or III of human clinical trials for various diseases and have been evaluated for toxicity, safety and bioavailability. This library thus offers an attractive set of drugs or drug-like molecules that have shown promising results in human patients. Each compound was tested in duplicate at 10 μM final concentration. 40% or more inhibition and 60% or more increase in hormone-binding activity of PR were used as cut-off values for a hit to be considered as an inhibitor or an activator, respectively. Out of 446 compounds tested, several primary hits were obtained (FIGS. 8A-8F). Inhibitors having chemical structures related to progesterone were not given further consideration, as they compete with 3[H]-progesterone for binding PR (FIG. 10 and Table 2). The remaining seven hits, which are structurally different from progesterone, were rescreened in triplicates at 20 μM final concentration (FIG. 9A, 9B and Table 1). Among these, only capsaicin showed a reproducible effect on blocking the hormonebinding activity of PR by about 40%. The hit rate is thus 0.22%, well within 1%, the maximum acceptable hit rate by NIH guidelines for a robust high-throughput drug-screening assay.

### Example 8

### Capsaicin Prevents 17AAG-Induced Overexpression of Hsp70 and Improves 17-AAG Cytotoxicity

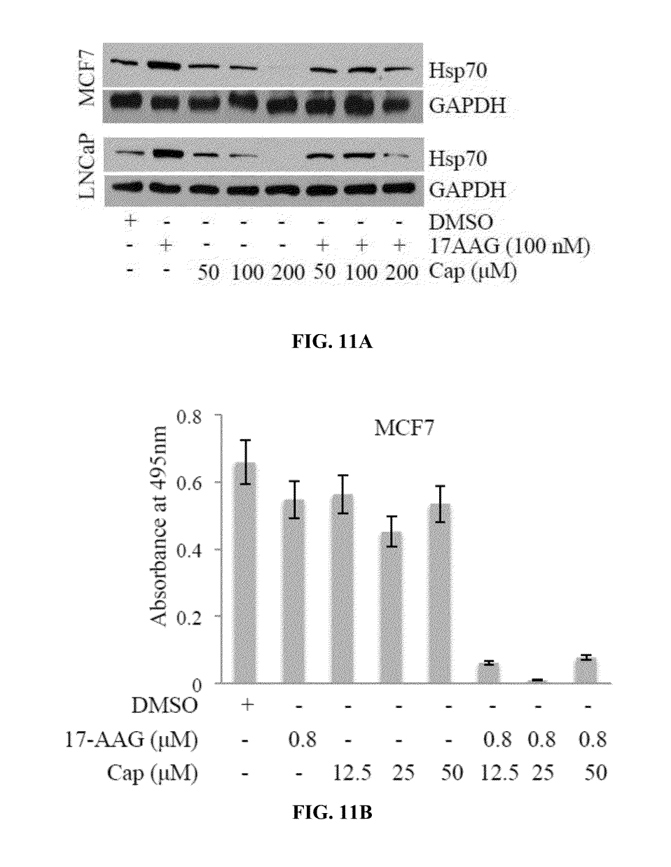

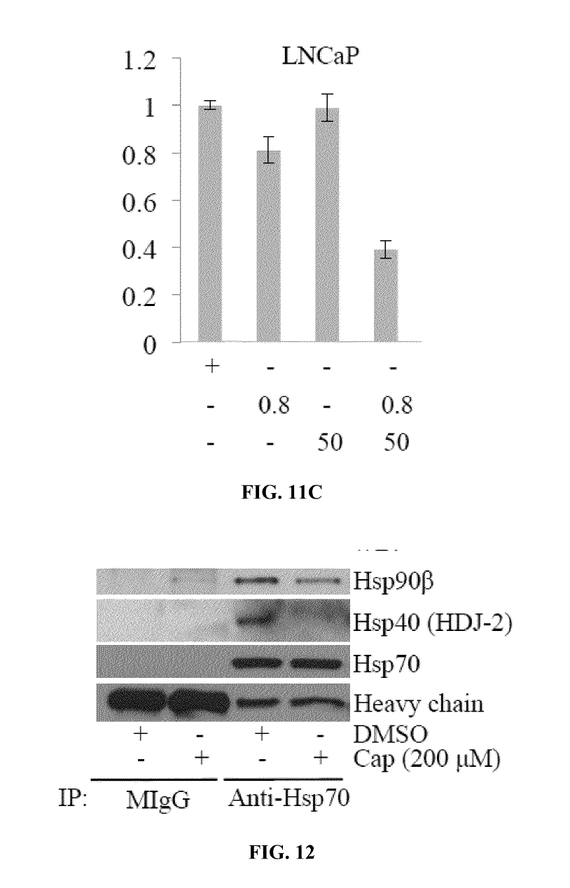

Pharmacological inhibition of Hsp90 by its N-terminal inhibitors such as 17-AAG is known to induce a heat shock response, which is thought to reduce the efficacy of these Hsp90 inhibitors through up-regulation of the antiapoptotic proteins Hsp70 and Hsp27. The ability of capsaicin to reduce the level of Hsp70 to prevent the 17-AAG-induced over-expression of the chaperone was tested. MCF7 and LNCaP cells were treated with 50, 100, and 200 μM capsaicin with or without 100 nM of 17-AAG. Excitingly, capsaicin blocked the over-expression of Hsp70 in the cotreated group as compared to monotherapy with 17-AAG (FIG. 11A). Cotreatment of capsaicin with 17-AAG was tested to determine if it would translate into an enhancement of 17-AAG's cytotoxic effect on cancer cells. As shown in FIG. 11B, MCF7 cells were treated for 48 h with DMSO control, 800 nM 17-AAG alone, or various concentrations of capsaicin (12.5, 25, 50 μM) or the combinations. As expected, cell survival analysis showed that monotherapy with 800 nM 17-AAG or with concentrations of capsaicin lower than 50 μM has minimal cytotoxicity. However, combination of capsaicin and 17-AAG leads to a powerful killing of MCF7. These results indicate that the dose of capsaicin required to achieve maximal efficacy could be reduced significantly. Similar data were observed with LNCaP cells where 800 nM 17-AAG combined with 50 μM capsaicin showed much more efficient killing compared to individual treatments (FIG. 11C). These data strongly demonstrate that capsaicin improves the 17-AAG's cancer cells cytotoxicity.

TABLE 1
Primary hits: Inhibitors and Activators (non-steroidal compounds)

| | | | Compound | |
|---|---|---|---|---|
| Number | Plate | Well Position | name | PubChem ID |
| 1 | NCP000685 | 4H | Zucapsaicin | 1548942 |
| 2 | NCP000685 | 3H | Progesterone | 5994 |
| 3 | NCP000685 | 8A | Cotinine | 854019 |
| 4 | NCP001097 | 3A | Tryptoline | 107838 |
| 5 | NCP001097 | 8A | Nafadotride | 3408722 |
| 6 | NCP001097 | 8F | AM-251 | 2125 |
| 7 | NCP000800 | 4A | Levosulpiride | 688272 |
| 8 | NCP000998 | 9A | Itopride HCL | 129791 |

TABLE 2
False positive hits (steroidal compounds)

| | | Well | | PubChem |
|---|---|---|---|---|
| Number | Plate | Position | Compound name | ID |
| 1 | NCP000685 | 10E | Ethinyl estradiol | 5991 |
| 2 | NCP001097 | 4C | Cortodoxone | 227112 |
| 3 | NCP001097 | 8B | Desoximethasone | 5753 |
| 4 | NCP001097 | 11D | Corticosterone | 444008 |
| 5 | NCP001097 | 11F | Tibolone | 9904 |
| 6 | NCP000800 | 2E | Nandrolone | 13109 |
| 7 | NCP000800 | 4G | Levonorgestrel | 6010 |
| 8 | NCP000800 | 7A | Methyltestosterone | 10633 |
| 9 | NCP000800 | 9G | Mestanolone | 11683 |
| 10 | NCP000800 | 10E | Megestrol Acetate | 216284 |
| 11 | NCP000899 | 6C | Zeranol | 229021 |
| 12 | NCP000899 | 7F | Methandriol | 3032303 |
| 13 | NCP000899 | 8D | Oxymetholone | |
| 14 | NCP000998 | 8C | Ioteprednol | |
| | | | etabonate | |
| 15 | NCP000998 | 11E | Mestranol | |
| 16 | NCP001169 | 6D | Stanozolol | |
| 17 | NCP001169 | 6G | Testosterone | |

### DISCUSSION

A new high-throughput drug-screening platform that is sensitive and robust enough to identify small molecule modulators of the Hsp90/Hsp70 chaperoning machinery in vitro was developed. This assay is uniquely qualified to identify compounds that inhibit as well as those which activate the Hsp90 chaperoning machine. Compounds that block the chaperoning activity of Hsp90/Hsp70 machinery can be further tested as cytotoxic molecules to treat cancer. Whereas compounds that potentiate the chaperoning activity of Hsp90/Hsp70 machinery can be used to treat neurodegenerative diseases such as Alzheimer's, Parkinson's, Huntington's disease where increased chaperoning activity of Hsp90/Hsp70 machinery might be used to reduce toxicity associated with misfolding of Hsp90/Hsp70 client proteins. This assay was used to screen a small NIH compound library and identified capsaicin as a hit. The data is the first evidence to show that capsaicin blocks the functioning of Hsp90/Hsp70 system in vitro and in cells. Treatment of several cancer cell lines with capsaicin resulted in dose dependent degradation of Hsp90 client proteins such as steroid receptors (AR, PR, GR) and signaling protein kinases (HER2, Akt, Raf, CDK4, Chk1) (FIG. 5A-C). Importantly both of these classes of clients are known to be pro-proliferative proteins that achieve their functional conformation by sequential steps of folding carried out by Hsp40, Hsp70, and HOP in the early stages of chaperoning. Hsp90, along with its co-chaperones such as p23 and Cdc37, carries out final stages of chaperoning of steroid receptors and signaling kinases, respectively.

Cell treatment with capsaicin selectively down-regulated the Hsp70 (inducible isoform) but not the Hsc70 (constitutive isoform). One other report has however shown that capsaicin induces overexpression of Hsp70 in HEK-293e kidney, MLE-12 lung. HT-29 gut and MCF7 breast cancer cell lines. At least in the common MCF7 cell line between the two studies, overexpression of Hsp70 at concentration ranging from 12.5 μM to 200 μM was not detected. Capsaicin has reproducibly caused degradation of Hsp70 in HeLa, LNCaP, MCF7, Hs578T and MDA-MB-231. This discrepancy between the data and other studies could be due to cell line differences but also to the fact that others looked at early time after capsaicin treatment (1 h), while most of this analysis was done at later time points (24 h). As shown in FIG. 12, after 6 h of treatment, the level of Hs70 is not significantly down (FIG. 12), but its functionality is compromised because normal Hsp70 chaperone complexes are altered (FIG. 12). Early overexpression of Hsp70 may reflect a transient capsaicin activation of heat shock factor 1 (HSF1) being dissociated from the Hsp90/Hs70 complex. However, prolong capsaicin treatment lead to Hsp70 protein degradation through lysosomal/autophagy pathway. HSF1 activation is manifested by increased Hsp70 mRNA level seen in capsaicin treated samples (FIG. 11A, lane 3), which confirms previous reports. It is well know that 17-AAG induce activation of HSF1 by dissociating it from the chaperone complexes. The data confirm this fact and further show that capsaicin and 17-AAG synergistically activate HSF1 as indicated by the large accumulation of Hsp70 mRNA (FIG. 11A, lane 4). However, Hsp70 protein is efficiently degraded, which deprive the cells from this sensor of the activation of the heat shock response and its pro-survival activities through inhibition of apoptosis.

The ability of capsaicin to selectively induce degradation of Hsp70 is remarkable and intriguing. The two Hsp70 isoforms share 85% amino acid sequence identity and their overall structures are remarkably similar. They however differ significantly in their carboxy-terminal domain (amino acids 510-641), which shows 70.5% amino acid identity. These differences between Hsp70 and Hsc70 could have a significant impact on their functions. Others measured the peptide binding affinities of purified Hsp70 and Hsc70 using three different radiolabelled peptides under a variety of buffer conditions in vitro. They found that Hsp70 binds with peptides with higher affinity than does Hsc70. Whether these differences play a role in capsaicin selectivity remain to be shown. Capsaicin has been shown to trigger several signaling events in a variety of cancer cell lines. The mechanism by which capsaicin induces apoptosis in cancer cells is not well understood, but it is clearly independent of the TRPV1 receptor. Indeed, neither capsazepine, a powerful vanilloid receptor antagonist, nor intracellular Ca²⁺ chelators significantly inhibited the capsaicin-induced apoptosis.

Capsaicin treatment induced mitophagy in MCF7 cells. Mitochondria in capsaicin-treated cells lost the healthy pattern of cristae as opposed to cells in the DMSO control group. The mitochondrial cristae constitute the inner mitochondrial membrane, which harbor protein complexes (complex I-V) of the electron transport system (ETS). Capsaicin treatment up-regulated p62/SQSTM1 levels in all cell lines tested (FIG. 3G). p62/SQSTM1 is an adapter protein that binds with autophagic substrate protein on one end and LC3 within the autophagosome membrane on the other end. p62/SQSTM1 gets degraded with its bound substrate by autophagy, and therefore accumulation of p62/SQSTM1 in cells is indicative of dysfunctional autophagy. Accumulation of p62/SQSTM1 protein in cells after capsaicin treatment indicates that although capsaicin induces autophagy, this autophagy may not be fully executed. Thus pro-survival features of autophagy maybe compromised leading to cancer cell death. How Hsp70 is degraded in an autophagy-dependent manner as indicated by rescue of Hsp70 upon 3MA treatment, is unknown. Interestingly, a recent report showed that over-expression of Hsp70 prevents starvation induced autophagy by up-regulation of mTOR-Akt pathway.

Hsp70 is a major anti-apoptotic protein known to be up-regulated in many cancers. 17-AAG and other N-terminal Hsp90 inhibitors cause over-expression of Hsp70 as a side effect. The data showed that co-treatment of capsaicin with 17-AAG prevented the 17-AAG-mediated upregulation of Hsp70, which translates into more potent cytotoxicity of MCF7 and LNCaP cells. These findings have relevance to clinical settings where the FDA-approved capsaicin could be used in combination with N-terminal Hsp90 inhibitors to induce a synergistic anti-tumor effect.

We claim:

1. A pharmaceutical composition comprising an effective amount of
a) a capsaicin, a synthetic capsaicin, or a derivative, analog or prodrug, or a pharmacologically active salt thereof to reduce, decrease, or inhibit the Hsp70; and
b) one or more inhibitors of Hsp90
to reduce, decrease, or inhibit the Hsp90 compared to a control.

2. The pharmaceutical composition of claim 1 wherein the one or more inhibitors of the Hsp90 pathway are selected from the group consisting of geldanamycin, tanspimycin (17-AAG), alvespimycin (17-DMAG), retaspimycin HCl (IPI-504), C-11, ganetespib (STA9090), SNX-2112, SNX-5542, NVP-AUY922, NVP-BEP800, CCT018159, VER-49009, PU3, BIIB021, herbimycin, derrubone, gedunin, celastrol (tripterine), (−)-epigallocatechin-3-gallate((−)-(EGCG), KW-2478, novobiocin, radicicol, radicicol oxime derivatives, radamide, radester, radanamycin, AT13387, debio0932, XL888 and pochonin A-F.

3. The pharmaceutical composition of claim 1 wherein one inhibitor of the Hsp90 pathway is tanespimycin (17-AAG).

4. The pharmaceutical composition of claim 1 further comprising a pharmaceutically acceptable excipient.

5. A blister pack comprising a plurality of dosage units comprising the pharmaceutical composition of claim 1.

6. A method for killing cancer cells or tumor cells comprising:
contacting the cancer cells or tumor cells with the pharmaceutical composition of claim 1.

7. The method of claim 6, wherein inducible Hsp70 is selectively inhibited in the cancer or tumor cells relative to constitutive Hsp70 expressed in the cancer or tumor cells.

8. The method of claim 6, wherein the contacting occurs in vivo in a subject in need of such treatment.

9. A method of treating cancer comprising administering to a subject with cancer an effective amount of the composition of claim 1 to kill cancer cells in the subject.

10. The method of claim 9 wherein the cancer is selected from the group consisting of lymphoma, B cell lymphoma, T cell lymphoma, mycosis fungoides, Hodgkin's Disease, myeloid leukemia, bladder cancer, brain cancer, nervous system cancer, head and neck cancer, squamous cell carcinoma of head and neck, kidney cancer, lung cancers such as small cell lung cancer and non-small cell lung cancer, neuroblastoma/glioblastoma, ovarian cancer, pancreatic cancer, prostate cancer, skin cancer, liver cancer, melanoma, squamous cell carcinomas of the mouth, throat, larynx, and lung, colon cancer, cervical cancer, cervical carcinoma, breast cancer including triple-negative breast cancer, epithelial cancer, renal cancer, genitourinary cancer, pulmonary cancer, esophageal carcinoma, head and neck carcinoma, large bowel cancer, hematopoietic cancers; testicular cancer; colon and rectal cancers, prostatic cancer including hormone-refractory prostate cancer and pancreatic cancer.

11. A method of inhibiting the Hsp70 and Hsp90 chaperone pathways in a cell comprising contacting one or more cells expressing the Hsp70/Hsp90 complex with an effective amount of a capsaicin, a synthetic capsaicin, or a derivative, analog or prodrug, or a pharmacologically active salt thereof in combination with one or more inhibitors of Hsp90 to selectively decrease or selectively inhibit inducible Hsp70 relative to constitutive Hsc70 and Hsp90 chaperone pathways in the cells compared to control cells.

12. The method of claim 10 wherein the naturally occurring capsaicin, synthetic capsaicin, or derivative, analog or prodrug, or pharmacologically active salt thereof in combination with one or more inhibitors of Hsp90 reduces the formation of, or increases the degradation of Hsp70 optionally including one or more co-chaperones or client proteins.

13. The method of claim 12 wherein the one or more client proteins is selected from the group consisting of AKT, pAKT and CDK4, ILK, Her2, Her3 and HOP.

14. The method of claim 11 wherein the capsaicin, synthetic capsaicin, or derivative, analog or prodrug, or pharmacologically active salt thereof in combination with one or more inhibitors of Hsp90 reduces or inhibits Hsp70 or Hsp90-mediated folding, activation, assembly, or function of proteins.

15. The method of claim 11 wherein the cells are under stress or transforming pressure.

16. The method of claim 11 wherein the cells are diseased or pathogenic.

17. The method of claim 11 wherein the capsaicin, synthetic capsaicin, or derivative, analog or prodrug, or pharmacologically active salt thereof in combination with one or more inhibitors of Hsp90 increases apoptosis of the contacted cells.

18. The method of 10 wherein the contact occurs in vivo in a subject in need thereof of.

19. The method claim 18 wherein in the subject has a disease or disorder selected from the group consisting of cancer, an inflammatory disease or disorder, a neurodegenerative disease, or an infectious disease.

20. The method of claim 19 further comprising administering to the subject one or more additional therapeutic agents.
